(19) United States
(12) Patent Application Publication
(10) Pub. No.: US 2023/0107688 A1
(43) Pub. Date: Apr. 6, 2023

(54) NEW NASAL RESPIRATORY APPARATUS

(71) Applicant: PNEUMA THERAPEUTICS, INC., Tucson, AZ (US)

(72) Inventor: David M. Kane, Windham, NH (US)

(21) Appl. No.: 17/906,039

(22) PCT Filed: Mar. 11, 2021
(86) PCT No.: PCT/US2021/021829

§ 371 Date: Sep. 9, 2022

Related U.S. Application Data
(60) Provisional application No. 62/987,944, filed on Mar. 11, 2020.
(60) Provisional application No. 62/992,333, filed on Mar. 20, 2020.
(60) Provisional application No. 63/006,407, filed on Apr. 7, 2020.
(60) Provisional application No. 63/006,411, filed on Apr. 7, 2020.

(51) Int. Cl. A61M 16/00; A61M 16/20; A61M 16/10

(52) U.S. Cl. CPC A61M 16/024; A61M 16/201; A61M 16/10; A61M 16/208; A61M 16/0003; A61M 2205/3334; A61M 2210/0618; A61M 2230/432; A61M 2016/0015

(57) ABSTRACT

A nasal ventilator assembly provides pressurized nasal oxygenation, ventilation and end tidal CO2 sampling with a movable piston, actuation of which opens and closes a vent to vent exhaled gasses to atmosphere. The piston is moved by force applied by gas supply and patient exhalation to a piston surface to actuate a spring to unblock the vent or vents to cause fluid communication between an air chamber and atmosphere. Also disclosed are an intelligent gas source and various valve configurations for use in the intelligent gas source for supplying gas to the pressurized nasal ventilator assembly or other nasal ventilation device.

### CROSS REFERENCE TO RELATED APPLICATIONS

This application is claims priority to U.S. Provisional Patent Application Ser. No. 62/987,944, filed Mar. 11, 2020, U.S. Provisional Patent Application Ser. No. 62/992,333, filed Mar. 20, 2020, U.S. Provisional Patent Application Ser. No. 63/006,407, filed Apr. 7, 2020 and U.S. Provisional Patent Application Ser. No. 63/006,411, filed Apr. 7, 2020, all pending, which applications are hereby incorporated by this reference in their entirety to the extent allowed by law.

### BACKGROUND

### Field

Embodiments of the present invention relate to oxygenation, ventilation, end tidal carbon dioxide (CO₂) sampling during general anesthesia and deep sedation, and specifically to a nasal respiratory platform with various related features.

### Background

General anesthesia has historically utilized a full-face mask attached to an anesthesia machine to support providing anesthetic gases and oxygen, as well as ventilating the patient and monitoring exhaled end tidal CO₂ levels. A major issue with using a full-face mask is that the mask must be removed for oral access to place an intubation tube, resulting in an apenic period. Respiratory compromise is a common result from the apenic period for high-risk patients.

Given the trend for more minimally invasive procedures, the use of intravenous deep sedation has grown significantly. Nasal cannula are used providing nasal oxygenation, but do not provide pressurization, sometimes resulting in respiratory compromise if the nasal pharynx becomes blocked.

Accordingly, there is a need for a nasal respiratory platform supporting pressurized nasal oxygenation, ventilation, and expired End Tidal CO₂ sampling by interfacing with and sealing about the nasal base of the nose.

A representative inhalation and exhalation cycle (flow and pressure provided by the ventilator) for a patient-ventilator interface during noninvasive ventilation is shown in FIG. 1. Inspiration of gas into the patient's lungs occurs when the flow rate as measured in L/min is positive while expiration occurs when the flow rate as measured in L/min is negative. Note that in this example, a minimum pressure of nominally 5 CM H₂O is maintained in order to provide Positive Expiratory End Pressure (PEEP). PEEP is a therapy provided in order to avoid passive emptying of the lung.

To address the shortcomings of full-face masks and nasal cannula, nasal ventilation masks covering the nose and sealing against the face are becoming popular. nasal ventilation masks support pressurization required to overcome blockage of the nasal pharynx, but obstruct the region near the eyes, easily lose a seal if the mask is tilted or if there is facial hair such as a mustache is present.

A nasal respiratory apparatus according to principles described herein and its various embodiments and combinations of features addresses the major shortcomings of all three of these approaches, supporting pressurized oxygenation, ventilation and end-tidal CO₂ sampling via nasal ventilation system that seals via the nares and nasal vestibule. This results in a more secure seal. The device is much more compact an unobtrusive than either mask approach, allowing for oral and eye access if required.

### BRIEF SUMMARY OF THE DISCLOSURE

Accordingly, the present invention is directed to nasal respiratory apparatus that obviates one or more of the problems due to limitations and disadvantages of the related art.

According to principles described herein, a nasal ventilator assembly includes a nasal interface comprising at least one opening for fluid communication with the nares of a patient; an air chamber in fluid communication with the nasal interface, a gas supply port and an end tidal sample port, a vent assembly comprising: a vent housing comprising at least one housing vent hole in fluid communication with ambient atmosphere and a piston assembly comprising a piston, a spring, a piston end wall operatively connected with the piston and the spring and a piston driven wall operatively connected with the piston and the spring. The piston driven wall includes at least one piston wall vent hole in fluid communication with the air chamber, wherein motion of the piston is caused by inhalation and exhalation of a patient to move the housing vent hole to align with the piston wall vent hole upon exhalation of the patient to cause the air chamber to be in fluid communication with the ambient atmosphere upon exhalation of the patient and to be fluidically sealed from the ambient atmosphere during inhalation of the patient.

In another aspect of the principles described herein, a gas source controller for pressurized oxygenation of a patient includes a valve assembly, comprising a valve gate assembly comprising a valve gate, an actuator assembly operatively connected to the valve gate to cause motion of the valve gate in a predetermined direction, a reservoir housing comprising a gas reservoir for containing a predetermined volume of gas therein, an end cap having a gas inlet therethrough, and an actuator control operatively coupled to the actuator assembly; an outlet assembly comprising a gas sensor and a gas source outlet; and a user interface for receiving control signals for driving the actuator assembly.

In another aspect, the valve gate may have a flat surface opposite the output assembly, and the valve gate assembly may further include comprising a reaction ring operatively interfacing with and fluidically sealed against the outlet assembly, one or more flexures operatively connected to the valve gate and the reaction ring such that forces generated by the gate motion react through the reaction ring to fluidically seal against the reservoir housing.

In another aspect, the valve assembly may include a heater. The gas controller may also include patient respiration sensor to sense the patient's respiration and to provide timing signals to the gas controller.

Further embodiments, features, and advantages of the nasal ventilator assembly, gas source controller, as well as the structure and operation of the various embodiments thereof, are described in detail below with reference to the accompanying drawings.

It is to be understood that both the foregoing general description and the following detailed description are exemplary and explanatory only, and are not restrictive of the invention as claimed.

### BRIEF DESCRIPTION OF THE DRAWINGS

The accompanying figures, which are incorporated herein and form part of the specification, illustrate a new nasal respiratory platform. Together with the description, the figures further serve to explain the principles of the new nasal respiratory platform described herein and thereby enable a person skilled in the pertinent art to make and use the new nasal respiratory platform.

FIG. 1 illustrates representative gas flow rate and pressure waveforms for an inspiratory and expiratory cycle.

FIG. 2 illustrates a pressurized nasal ventilator assembly in a respiratory system.

FIG. 3 illustrates a pressurized nasal ventilator assembly according to principles described herein.

FIG. 4 illustrates a pressurized nasal ventilator assembly inspiratory and expiratory configurations according to principles described herein.

FIG. 5 is an exploded view of a pressurized nasal ventilator assembly according to principles described herein.

FIG. 6 illustrates details of an exemplary piston assembly according to principles described herein.

FIG. 7 illustrates details of an exemplary vent housing according to principles described herein.

FIG. 8 illustrates a pneumatic circuit for a pressurized nasal ventilator assembly according to principles described herein.

FIG. 9 illustrates operation of an embodiment of a pressurized nasal ventilator assembly according to principles described herein.

FIG. 10 illustrates relative facial obstruction for an embodiment of a pressurized nasal ventilator according to principles described herein.

FIG. 11 illustrates vent flow resistance during the inspiratory/expiratory cycle.

FIG. 12 illustrates another embodiment of a pressurized nasal ventilator assembly according to principles described herein.

FIG. 13 illustrates operation of the embodiment of a pressurized nasal ventilator assembly of FIG. 12.

FIG. 14 illustrates another embodiment of a pressurized nasal ventilator assembly according to principles described herein.

FIG. 15 illustrates operation of the embodiment of a pressurized nasal ventilator assembly of FIG. 14.

FIG. 16 illustrates another embodiment of a pressurized nasal ventilator assembly according to principles described herein.

FIG. 17 illustrates operation of the embodiment of a pressurized nasal ventilator assembly of FIG. 16.

FIG. 18 illustrates an intelligent gas source supplying gas to a pressurized nasal ventilator assembly according to principles described herein.

FIG. 19 illustrates details of the intelligent gas source of FIG. 18.

FIG. 20 illustrates a vent assembly and actuator on a high pressure side of a valve gate for use in an intelligent gas source according to principles described herein.

FIG. 21 illustrates an actuator assembly for use in an intelligent gas source according to principles described herein.

FIG. 22 illustrates a spring assembly for use in an intelligent gas source according to principles described herein.

FIG. 23 illustrates a section view of a valve assembly according to principles described herein.

FIG. 24 illustrates gas flow through a general model for an outlet assembly and a valve gate of an intelligent gas source according to principles described herein.

FIG. 25 is a generalized flow circuit model based on flow between parallel plates.

FIG. 26 illustrates an example intelligent gas source system according to principles described herein.

FIG. 27 illustrates an alternate valve assembly for use in an intelligent gas source according to principles described herein.

FIG. 28 is a section view of the alternate valve illustrated in FIG. 27.

FIG. 29 is a model for illustrating force and momentum balance for a generalized valve gate according to principles described herein.

FIG. 30 illustrates an exemplary valve assembly with an actuator on a low pressure side of the valve gate for an intelligent gas source according to principles described herein.

FIG. 31 is a section view of an example valve assembly with an actuator on a low pressure side of the valve gate for an intelligent gas source according to principles described herein.

FIG. 32 illustrates an athermal valve assembly with an actuator on a low pressure side of the valve gate for an intelligent gas source according to principles described herein.

FIG. 33 is a section view of an athermal valve gate with an actuator on the low pressure side of the valve gate for use in an intelligent gas source according to principles described herein.

FIG. 34 shows a comparison of an actuator gap resulting from a temperature increase for an all stainless steel and athermal valve configuration according to principles described herein.

FIG. 35 illustrates a simplified actuator valve assembly according to principles described herein.

FIG. 36 illustrates a simplified three actuator valve assembly according to principles described herein.

FIG. 37 illustrates a simplified 2-stage actuator valve assembly according to principles described herein.

FIG. 38 illustrates another embodiment of an athermal valve assembly according to principles described herein.

FIG. 39 is a section view of an embodiment of an athermal valve assembly according to principles described herein.

FIG. 40 illustrates an embodiment of a valve gate assembly according to principles described herein.

FIG. 41 illustrates a valve assembly with μgap heater according to principles described herein.

FIG. 42 illustrates an intelligent gas source system with patient synchronicity sensor according to principles described herein.

FIG. 43 illustrates inhalation and exhalation motion and pressurized controlled ventilation waveforms according to principles described herein.

FIG. 44 illustrates inhalation and exhalation motion and volume controlled ventilation waveforms according to principles described herein.

### DETAILED DESCRIPTION

Reference will now be made in detail to embodiments of the new nasal respiratory apparatus with reference to the accompanying figures. For convenience of explanation, various figures make use of a right-handed X, Y, Z-axis Cartesian Coordinate system reference space, with reference to X-Y, X-Z, and Y-Z planes.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

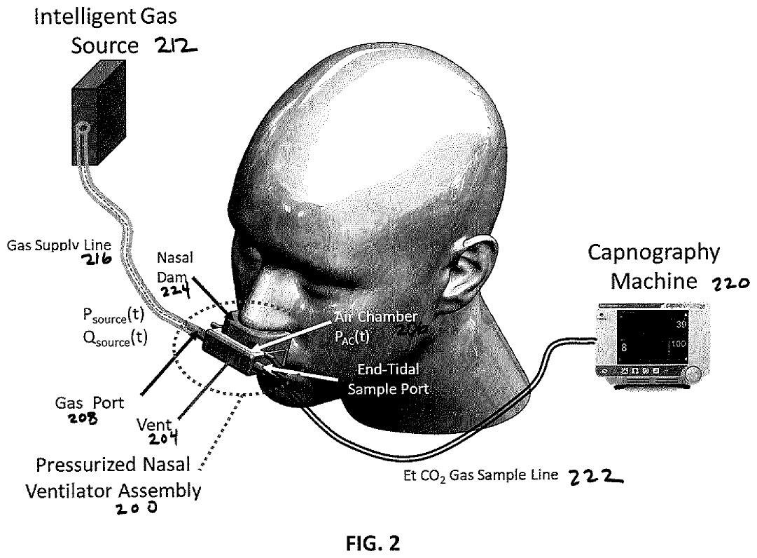

FIG. 2 illustrates an example pressurized nasal ventilator assembly 200 as applied to a patient according to principles described herein. As shown in FIG. 2, the pressurized nasal ventilator assembly 200 includes a vent 204 into an air chamber 206, a gas port 208 connected to a gas supply 212 via a gas supply line 216, and an end tidal sample port 218 connected to a capnography machine 220 via an end tidal gas sample line 222. The pressurized nasal ventilator assembly 200 further includes a “nasal dam” 224 as a substantially sealed interface to the patients nares. The nasal dam as shown herein is described in detail in PCT Application No. PCT/US2021/013192, filed Jan. 13, 2021, and claiming priority to U.S. Provisional Patent Application Ser. No. 62/960,385, filed Jan. 13, 2020, the entirety of these application hereby incorporated by reference for all purposes as if fully set forth herein to the extent allowed by law. While illustrated herein as a nasal dam, it should be appreciated by those of skill in the art that any nasal interface that provides a substantially sealed interface between the pressurized nasal ventilator assembly 200 and the patient's nares/nostrils could be used in the disclosed pressurized nasal ventilator assembly 200 within the scope of the present disclosure.

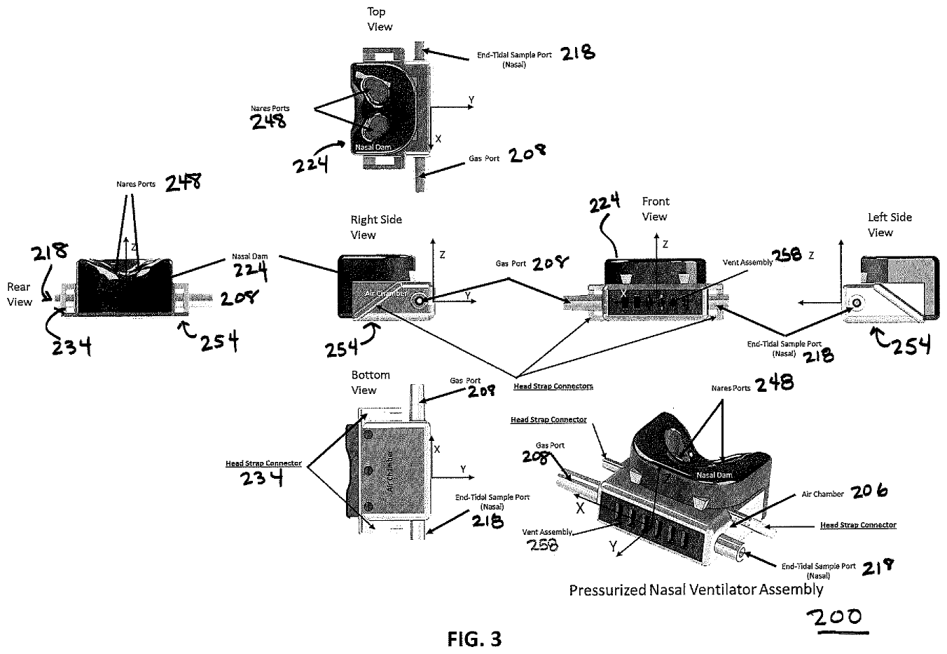

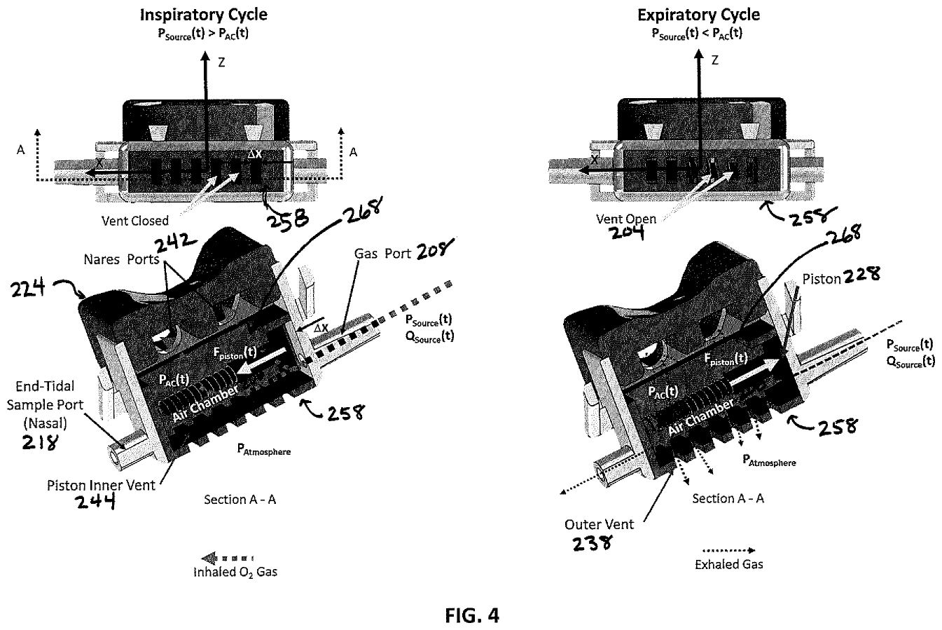

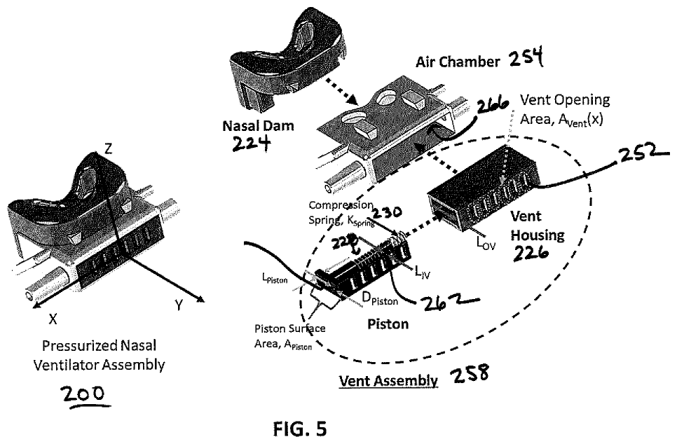

FIG. 3 illustrates a pressurized nasal ventilator assembly 200 according to principles described using a nasal dam 224. FIG. 4 is a cross-sectional view of a pressurized nasal ventilator assembly 200 according to principles described herein using a nasal dam 224. FIG. 5 is an exploded view of the pressurized nasal ventilation assembly 200 using the nasal dam 224.

Looking to FIG. 5, it can be seen for the purposes of description, the pressurized nasal ventilation assembly 200 includes three parts: a nasal dam 224 or other appropriate nasal interface, an air chamber assembly 254 forming the air chamber 206 therein, and a vent assembly 258. As discussed earlier, the nasal dam 224 is described in detail in PCT Application No. PCT/US2021/013192, filed Jan. 13, 2021, but that the pressurized nasal ventilator assembly according to principles described herein is not limited to the nasal dam configurations disclosed herein or disclosed in PCT Application No. PCT/US2021/013192.

As can best be seen in FIG. 5, the air chamber assembly 254 includes an air chamber 206, gas and end tidal sample ports (208, 218) in fluid communication with the air chamber 206 and nares ports 242 through an upper wall 260 that correspond to nares ports in the nasal dam 224 that provide fluid communication between the air chamber and the patient's nostrils. In the present example using the previously disclosed nasal dam 224, the nasal dam is inserted to the rear of the air chamber assembly 254, thus enclosing the rear of the air chamber assembly 254 to provide at least one wall forming the air chamber 206.

The nasal dam as used herein surrounds the air chamber nares ports 242 and interfaces with the soft tissue of the Nasal base, providing a pressure seal in order to contain airflow between the nasal pharynx and the nasal ventilator assembly. The nasal dam may be of a soft Shore A 5-20 durometer material in order to conform to and seal the nasal base from a pressure differential between the air chamber interior and the atmosphere.

Referring again to FIG. 5, the vent assembly 258 includes a vent housing 226, a movable piston-driven wall 262, a piston 228 and a compression spring 230. The piston-driven wall 262, the piston and the compression spring form a piston assembly 264 that fits within a cavity 266 formed by the vent housing 226. Upon insertion of the piston assembly 264 into the cavity 266, the piston-driven wall 248 is adjacent a housing front wall 240 of the vent housing 226. The vent assembly 258 with the piston assembly 264 in the cavity 266 fits within the air chamber 206, as illustrated in FIGS. 3 and 4. As can be seen in FIG. 4, the vent housing 226 on a side proximate the rear of the air chamber assembly 254 (upon insertion into the air chamber 206) may have a back wall or back wall portion 268 so as to allow air flow from the nares ports into the air chamber 206 and to the interior of the vent housing 226. The back wall or back wall portion 268 thus has an air chamber opening 272 at the side proximate the nasal dam 224. As can be appreciated by one of skill in the art, the vent housing may have no back wall portion 268 and still fall within the scope of the present disclosure. Upon insertion into the air chamber assembly 254, the vent assembly 258 substantially seals the air chamber 206 from the exterior atmosphere or allows ventilation to the exterior atmosphere from the air chamber 206 according to operational principles described herein (but for the nares ports, end tidal sample ports and the gas ports, which would allow air flow into the air chamber until connected to the patient, the capnography sensor, and the gas supply, respectively).

The air chamber assembly 254 with the vent assembly 258 therein is inserted into the front of the nasal dam 224. In some embodiments, the back wall of the air chamber 206 may be a rear wall of the air chamber assembly (not shown) or provided by the fit of a portion of the nasal dam 224 into a rear of the air chamber assembly, as illustrated in FIG. 4.

The pressurized nasal ventilator assembly thus includes a gas connection port 208, which provides an interface with an intelligent gas source, to be described later, or other gas source. The gas connection port 208 allows for supply of ventilation gas such as oxygen (02) via the gas supply line 216. The gas connection port 208 may be designed to comply with appropriate connection standards, such as comply to the dimensions specified for a Test Nipple SO/DIS 17256 Anaesthetic and respiratory equipment. Other gas port configurations are possible. The end tidal sample port 218 may be designed to comply with ISO 80396-7: 2016€, for the Nominal Dimensions. While shown as extending in a direction parallel to the X-axis of the figures, one of skill in the art would appreciate that the gas connection port and the end tidal sample port may extend in any appropriate direction. The assembly 200 may further include head strap connector points 234.

The air chamber 206 provides a structural and gas flow interface between the gas supply tube 216, nares ports 242 of the air chamber 206 and the nares ports of the nasal dam 224, and the end tidal sample port 218.

The vent assembly 258 includes the piston assembly 264, which houses the piston 228 and the compression spring 230, within vent housing 226. The vent housing 226 includes the housing front wall 240 distal to the rear of the air chamber assembly 254 and includes a plurality of housing vent openings 238 therethrough. In the piston assembly 264, the piston 228 is operatively connected to piston-driven front wall 262 that includes a piston inner vent 244 comprising a plurality of piston (inner) vent openings 252 that correspond with the housing (outer) vent openings 238 such that during operation of the piston 228, there is a state in which the piston vent openings 252 are caused to align with the housing vent openings 238 to cause fluidic communication with the interior of the air chamber 206 with the exterior atmosphere.

The piston 228 is designed to slide within the vent housing 226 and the piston assembly 264 to cause the piston-driven wall 262 to move within the vent housing 254 to block or close the housing vent openings 238 by causing the piston-driven wall to align with the housing front wall 240 in such a way to block all or in part fluid communication between the air chamber 206 and the exterior atmosphere that might otherwise be possible through the housing vent openings 238. The compression spring 230 sets a force level required for the piston 228, when pressurized, to slide, blocking or closing the housing (outer) vent openings 238 or, conversely restore the piston 228 to an “open” position in which the piston vent openings 252 align with the housing vent openings 238 when the adequate pressure gradient is removed/decreased.

FIG. 4 illustrates details of an exemplary embodiment of the pressurized nasal ventilator assembly and airflow therethrough. In the illustrated embodiment of the pressurized nasal ventilator assembly of FIG. 4, the vent is driven during the inspiratory phase to a closed position relative to the atmosphere due to the force on a ventilation assembly piston due to the pressure difference between P_{Source} and P_{AC}. The result is that the inspiratory flow Q_{Gas}, is forced through the nares and ultimately into the lungs. During the expiratory phase when gas is flowing from the lungs, Q_{Expiratory}, into the air chamber 206, Q_{Gas} is reduced in order for the pressure in the air chamber 206 P_{AC} to be greater than P_{Source}, resulting in a force that drives the piston-driven wall 262 such that inner vent openings 252 are aligned with outer vent openings 238 so that the air chamber is opened to the atmosphere, allowing the expiratory gases to leave the air chamber to the atmosphere.

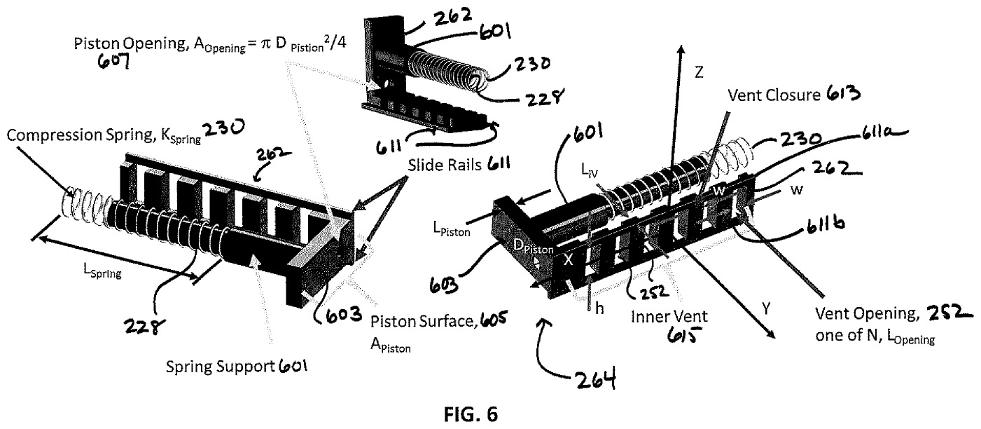

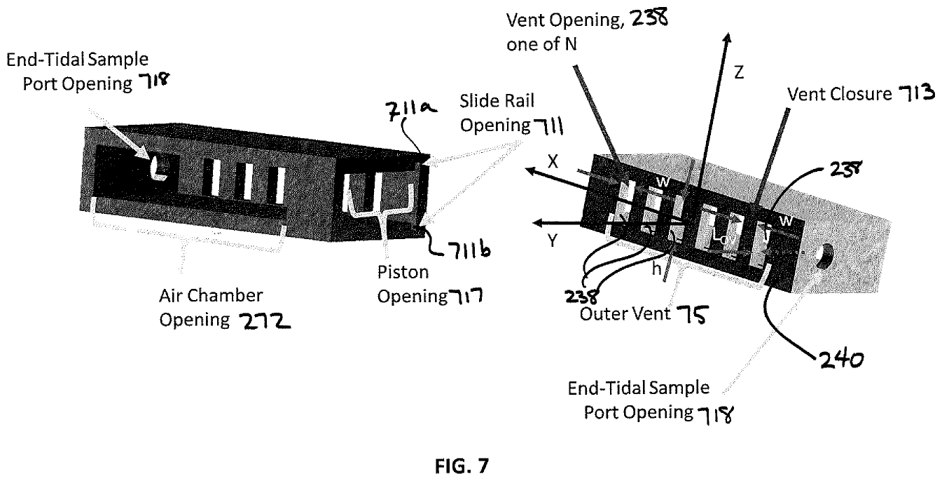

FIG. 6 illustrates details of an example embodiment of the piston assembly 264. FIG. 7 illustrates details of an example embodiment of the vent housing 226.

Referring to FIG. 6, the piston assembly 264 includes the piston 228, the compression spring 230 on the piston 228, a spring support 601 extending from an end of the piston 228, a piston end wall 603 at an end of the spring support 601 opposite the piston. The spring support 601 extends from the back side of the piston end wall 603 and constrains a compression spring 230 along the Y and Z axis, allowing for compression of the spring 230 and motion along the X axis. The piston end wall 603 operatively connects the piston/compression spring/spring support assembly 609 to the piston-driven wall 262 and having a piston surface 605 that receives or captures gas flowing from the gas connection port 208 in the air chamber assembly 254 such that the gas flowing from the gas connection port 208 pushes against the piston end wall 603 to move the piston end wall 603 to actuate the compression spring 230 and move the piston 228. The piston surface 605 has an area A_{Piston}.

The piston end wall 603 further includes a piston opening 607 therethrough. The piston opening 607 is concentric with the gas connection port 208 opening of the air chamber assembly 254 and allows for gas flow from the gas connection port 208 into the air chamber 206 through the piston end wall 603. The piston opening has an opening diameter D_{Piston}, with area A_{Opening} in the Y-Z plane and a length parallel to the X axis of L_{Piston}.

The piston-driven wall 262 is substantially parallel to the piston/compression spring/spring support assembly 609 (in the X-direction of the coordinate system of the figures). The piston-driven wall 262 includes two slide rails 611 including a top slide rail 611a and a bottom slide rail 611b. In operation the top and bottom slide rail 611a and 611b travels in a slide rail opening or groove 711 (FIG. 7) of the vent housing 226. Accordingly, motion of the piston 228 relative to the vent housing 226 is constrained in all degrees of freedom with the exception of travel parallel to the X axis. The piston-driven wall 262 includes an inner vent 615 that includes N piston (inner) vent openings 252 with dimensions ΔX parallel to the X axis and L_{IV} parallel to the Z axis. The openings alternate with corresponding vent closure surfaces 613 ΔX wide along the X axis so that when there is no piston displacement along the −X axis, the piston (inner) vent openings 252 of the piston-driven wall 262 and the vent housing (outer) openings 238 are aligned, and the vent 204 is open pneumatically to the atmosphere such that the air chamber 206 is open to the atmosphere. When the pressure P_{Source} results in a force in the −X direction that is greater than F_{PL} plus K_{Spring} ΔX, the piston moves by ΔX in the −X direction and the openings of the piston Inner Vent openings are aligned with the vent closures 713 of the housing front wall 244 and the air chamber 206 interior is pneumatically isolated from the atmosphere.

Referring to FIG. 7, the vent housing 226 includes an outer vent 715 in the outer front wall 240 and including N housing (outer) vent openings 238 with dimensions ΔX parallel to the X axis and L_{IV} parallel to the Z axis. The outer vent openings 238 alternate with corresponding vent closure surfaces 713 ΔX wide along the X axis so that when there is no piston displacement along the −X axis the openings of the inner vent openings 252 and the outer vent openings 238 are aligned and the vent 204 is open pneumatically to the atmosphere. The vent housing 226 further includes a piston opening 717 into which the piston assembly 264 is inserted and the air chamber opening 272. The vent housing 226 further includes slide rail openings 711a and 711b adjacent a side of the piston opening 717 corresponding to where the piston-driven wall 262 will be located within the vent housing 226 upon insertion of the piston assembly 264 in the piston opening 717. Corresponding with the slide rails 611a and 661b, the top and bottom slide rail openings 711a and 711b run parallel to the X axis to contain the piston slide rails 611a/611b in the Y-Z direction but allowing for motion along the X axis. The vent housing 226 further includes an end tidal sample port opening 718 is concentric with the end tidal sample port 218 of the air chamber 206, thus allowing for the passage of exhaled gas from the air chamber 206 to the end tidal sample port 218.

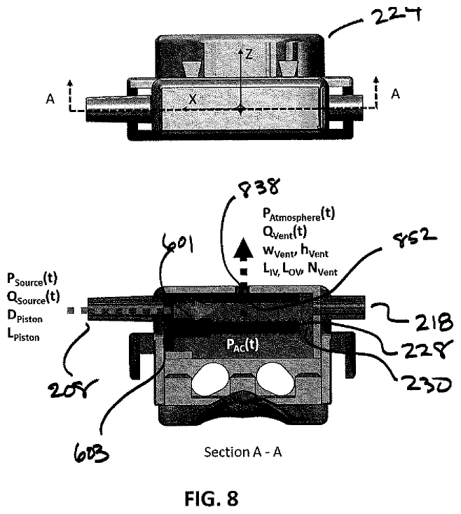

Flow into the air chamber 206 through the piston opening 607 will generally be turbulent with a Reynolds Number (Re) >4000. The pressurized nasal ventilator assembly can be modeled as a pneumatic circuit illustrated in FIG. 8. Flow from a gas source into the air chamber 206 is governed by Equation (5), below, where:

Equations governing Pressure Difference P_{Source}(t)−P_{AC}(t):

P_{Source}(t) is the Gas Source Pressure (force per unit area) as a function of time, t.

P_{AC}(t) is the Air Chamber pressure (force per unit area) as a function of time, t.

Q_{Source}(t) is the Gas Source Flow Rate (volume per unit time) as a function of time, t.

$${R}_{Piston} = Piston flow resistance , kg / {m}^{7} \qquad ( 1 )$$

$${D}_{Hydraulic} = Hydraulic Diameter , m$$

$$= {D}_{Piston} for a circular tube$$

$$µ = Dynamic Viscosity , kg \cdot / m - {}^{.} s$$

$$ρ = Gas Density , kg / {m}^{3}$$

$${V}_{Avg} = Average gas velocity$$

$$= {Q}_{Source} ( t ) / π {D}_{Hydraulic}^{2} / 4$$

$$Re = Reynolds Number \qquad ( 2 )$$

$$= ρ {V}_{avg} {D}_{Hydraulic} / µ$$

$$ε = Absolute Roughness , m$$

$$f = Friction factor ( Colebrook equation ) \qquad ( 3 )$$

$$= {[ - 2. log \{ 2.51 / {Ref}^{0.5} + ( ( ε / {D}_{Hydraulic} ) / 3.7 ) 1.11 \} ]}^{- 2}$$

$${R}_{Piston} = 8 {fL}_{Piston} ρ / {π}^{2} {D}_{Hydraulic}^{5} \qquad ( 4 )$$

$${P}_{Source} ( t ) - {P}_{AC} ( t ) = {{Q}_{Source} ( t )}^{2} {R}_{Piston} \qquad ( 5 )$$

Flow from the Air Chamber to the atmosphere is based on the geometry shown in FIGS. 6 and 7, governed by Equation 5, where:

P_{Atmosphere} is the atmospheric pressure (force per unit area).

Q_{Vent}(t) is the gas flow rate from the Air Chamber to the atmosphere (volume per unit time)

$$N = Number of vent openings \qquad ( 6 )$$

$$w = Vent opening width , m$$

$$h = Vent opening height , m$$

$${L}_{IV} = Inner vent opening length , m$$

$${L}_{OV} = Outer Vent Opening Length , m$$

$${L}_{Vent} = {L}_{IV} + {L}_{OV}$$

$${R}_{iVent} = Vent flow resistance , kg / {m}^{7}$$

$${D}_{Hydraulic} = Hydraulic Diameter , m$$

$$= 4 ( Nwh ) / ( N ( 2 w + 2 h ) for a rectangular tube ( see FIG . 7 ) Error ! Reference source not found .$$

$$= 2 wh / w + h$$

$${V}_{Vent} ( t ) = Gas velocity through vent , m / s \qquad ( 7 )$$

$$= {Q}_{Vent} ( t ) / Nwh$$

$${P}_{AC} ( t ) - {P}_{Atmosphere} = {fL}_{Vent} {ρ ( {Q}_{Vent} ( t ) / Nwh )}^{2} / ( 2 ( 2 wh / w + h ) ) \qquad ( 8 )$$

$$= {{Q}_{Vent} ( t )}^{2} {fL}_{Vent} ρ ( w + h ) / 4 {{N}^{2} ( wh )}^{3}$$

$${R}_{Vent} = {fL}_{Vent} ρ ( w + h ) / 4 {{N}^{2} ( wh )}^{3} \qquad ( 9 )$$

$${P}_{AC} ( t ) - {P}_{Atmosphere} = {{Q}_{Vent} ( t )}^{2} {R}_{Vent} \qquad ( 10 )$$

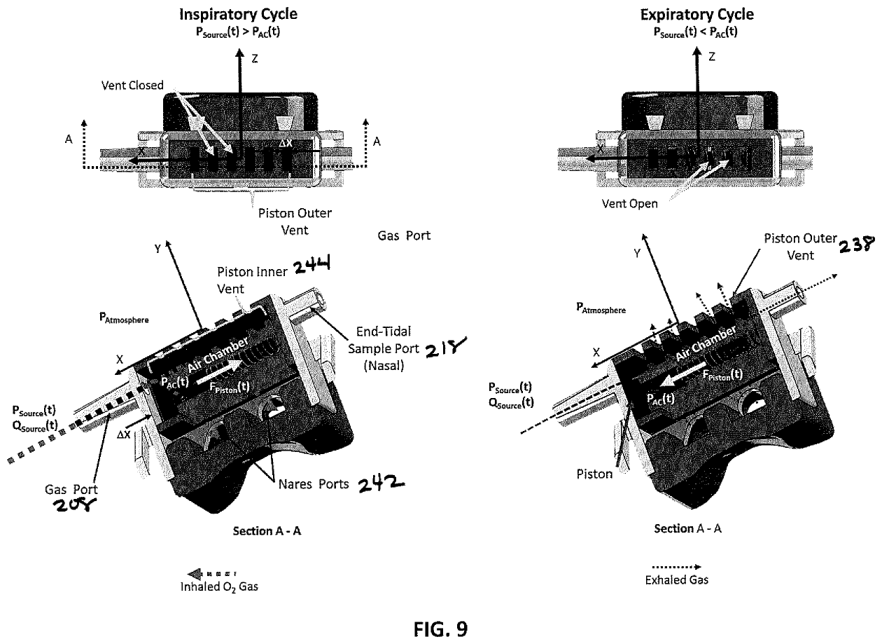

Detail of the Pressurized Nasal Ventilation Assembly is illustrated in FIG. 9. The piston is driven along the x axis of the ventilation housing by the pressure differential between the Intelligent Gas Source pressure, P_{Source}(t) and the pressure in the air chamber, PAC(t) across the piston surface having an area A_{Piston}(t). When in its initial non-displaced position, the spring on the piston has been preloaded so there is an initial preload force, F_{PL}, in the plus X direction. The analysis leading to the force balance equation between the spring force on the piston in the plus X direction and the pneumatic forces on the piston are shown in Equations 11-13.

Compression spring constant (force per unit distance compressed), K_{Spring}

Preload compression distance, ΔX_{PL}

Spring preload force, F_{PL}

F_{PL}=K_{Spring}ΔX_{PL} (11)

Source Pressure, P_{Source}(t)

Source flow rate, Q_{Source}(t)

Air Chamber pressure, P_{AC}(t)

Piston surface area, A_{Piston}

Force due to fluid flow through piston opening, F_{Flow}(t)

$${F}_{Flow} ( t ) = ( {P}_{Source} ( t ) - {P}_{AC} ( t ) ) {A}_{Piston} \qquad ( 12 )$$

$$= {{Q}_{Source} ( t )}^{2} {R}_{Piston} {A}_{Piston}$$

The Piston will move ΔX, completely closing the vent when the following condition is met:

F_{Flow}(t)>F_{PL}+K_{Spring}ΔX (13)

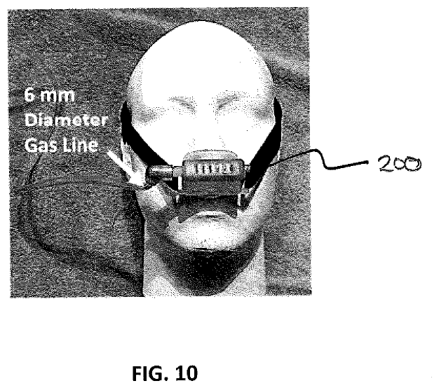

FIG. 10 shows a nasal ventilator assembly according to principles described herein as would be worn by a patient.

The pressurized nasal ventilator assembly provides a significant benefit over nasal cannula or high flow nasal cannula currently used for non-invasive ventilation for patients with respiratory compromise. Neither cannula design can overcome an upper airway obstruction for the static pressure is typically no greater than 5 CM H₂O where up to 15 CM H₂O can be required to overcome the obstruction. Pressurized nasal ventilation minimizes the potential for upper airway obstruction for pressures greater than 20 CM H₂O can be obtained. The pressurized nasal ventilation assembly has a reduction in hardware and the associated circuit size that must be connected to the patient to support the oxygenation and ventilation functions.

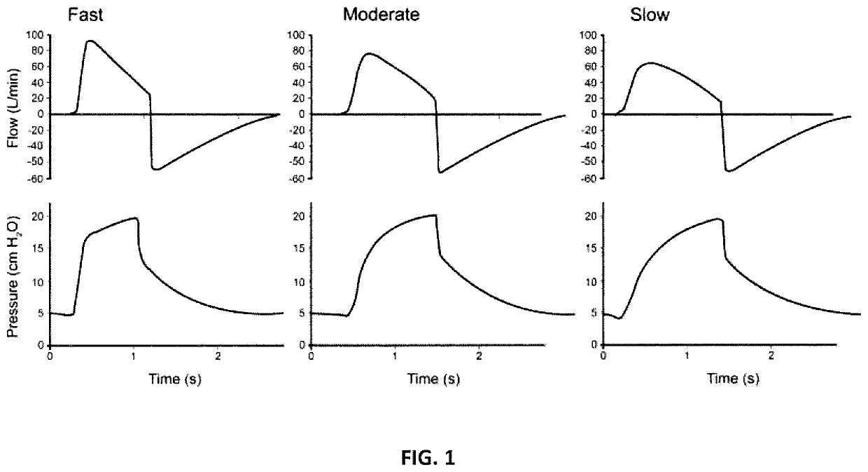

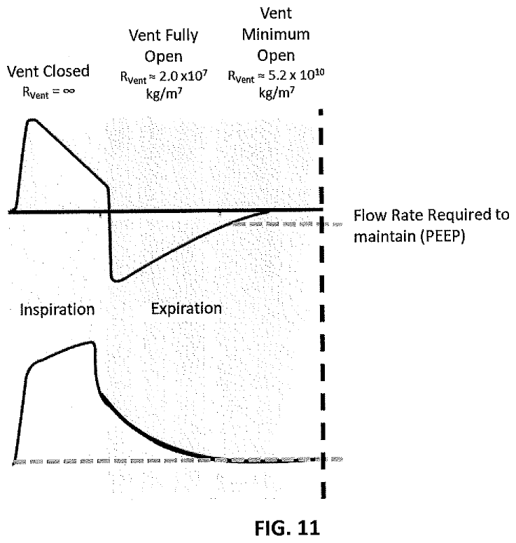

The vent assembly shown in FIG. 9 is a Binary Design, effectively open or closed depending on the resulting Piston, F_{Piston}, force due to the difference between the air chamber and source pressure, P_{Source}(t)−P_{AC}. As a result, the flow resistance from the Air Chamber through the Vent Assembly to the atmosphere is either infinite, or nearly zero. This having nearly zero flow resistance makes it difficult to maintain a PEEP pressure of approximately 5 CM H₂O as illustrated in FIG. 1. It may be desirable to have a variable vent flow resistance depending on where in the time in the inspiratory or expiratory cycle of the patient as illustrated in FIG. 11. This figure has replotted the “Fast” breathing cycle shown in FIG. 1 where the Inspiratory Phase begins at t=0.0 seconds through 0.9 seconds. The Vent resistance between the Air Chamber and atmosphere, R_{Vent}, is infinite at this point for the gas flow from the Intelligent Gas Source to enter the nares port and ultimately the lungs.

Expiratory flow then begins at t=0.9 seconds where the vent resistance is relatively low, R_{Vent}, ≈2.0×10⁷ kg/m⁷) for example. This allows the expiratory flow from the lungs to enter the air chamber and out the vent to the atmosphere. As the flow rate drops, the air chamber pressure also drops. If the PEEP level of 5 CM H₂is to be maintained, the vent flow resistance must be increased and gas from the Intelligent Gas Source is required in order to maintain the PEEP level. Nominally from time t=2.1 seconds to 3.0 seconds the vent resistance is increased to R_{Vent}, ≈5.2×10¹⁰ kg/m⁷) with the intelligent Gas Source providing 10 L/min. After 3.0 seconds, the Inspiratory portion of the cycle repeats.

In addition to the previously disclosed vent configurations, additional variable Vent resistance pressurized Nasal Ventilator configurations/exemplary embodiments are described herein. Principles regarding operation of the piston described above is not repeated in detail, only vent configurations.

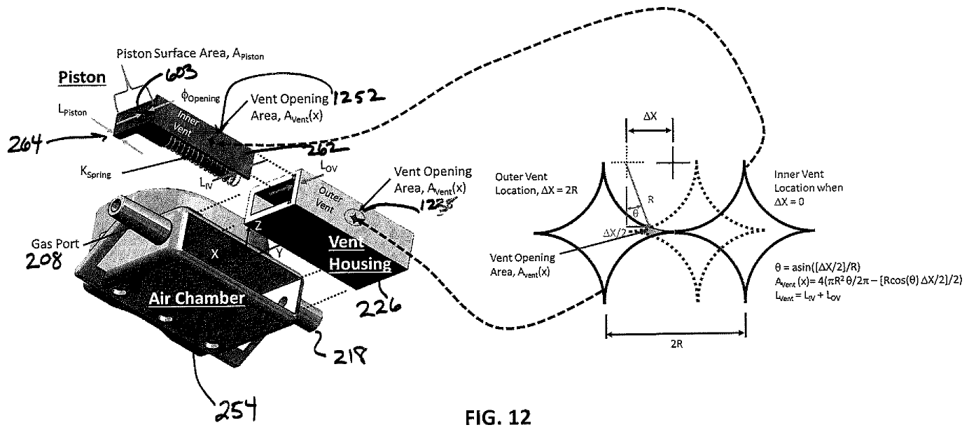

FIG. 12 illustrates an embodiment of the pressurized nasal ventilator having a vent opening with a “Star” shape, having an area A_{Vent}(x), that is a function of the piston position along the X axis inside the Vent Housing. The benefit of this design over the design illustrated in FIG. 9 is that flow control required to position the piston has a much looser tolerance. Positioning for the Star design can be of the order of +/−0.5 mm where design of FIG. 9 positioning tolerance is approximately ⅕^{th} of that.

The vent “Star” is composed of four quarter circle arcs with radius R. R_{Vent}(x) is calculated in Equations 14-21 as follows:

R=arc radius (14)

Angle between top of inner Vent arc and Inner Vent-Outer vent arc intersection point, θ.

θ(x)=a sin([ΔX/2]/R) (15)

Opening area of Vent, A_{Vent}(x)

A_{Vent}(x)=4(πR²θ/2π−[R cos(θ)ΔX/2]/2) (16)

Total length of vent, L_{Vent}

Inner vent length, L_{IV}

Outer vent length, L_{OV}

L_{Vent}=L_{IV}+L_{OV} (17)

P_{Atmosphere} is the atmospheric pressure (force per unit area).

P_{AC}(t)=Pressure in air chamber

Q_{Vent}(t) is the gas flow rate from the Air Chamber to the atmosphere (volume per unit time)

$${R}_{iVent} = Vent flow resistance$$

$$p ( x ) = Wetted perimeter as a function of x \qquad ( 18 )$$

$$= 4 ( 2 π R ) θ ( x ) / 2 π$$

$${D}_{Hydraulic} ( x ) = Hydraulic Diameter as a function of displacement in x \qquad ( 19 )$$

$$= 4 {A}_{Vent} ( x ) / p ( x )$$

$${R}_{Vent} ( x ) = 8 {fL}_{Vent} ρ / {π}^{2} {D}_{Hydraulic}^{5} \qquad ( 20 )$$

$${P}_{AC} ( t ) - {P}_{Atmosphere} = {{Q}_{Vent} ( t )}^{2} {R}_{Vent} ( x ) \qquad ( 21 )$$

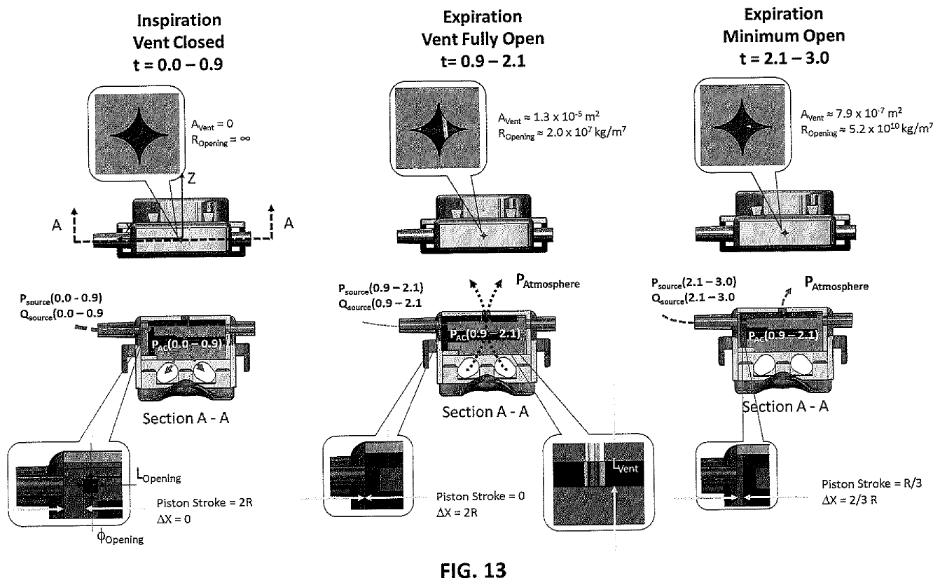

FIG. 13 illustrates operation of the embodiment of FIG. 12 during the breathing cycle. FIG. 13 shows the piston positioned along the X axis, thus the associated vent area, A_{Vent}(x), and resistance, R_{Vent}(x), for the inspiratory and expiratory cycle as a function of time in seconds. During the Inspiratory phase of the cycle, t=0 to t=0.9 seconds, the Intelligent Gas Source provides the required flow, Q_{Source}(t), thorough the gas port and the resulting force due to th pressure difference between the P_{Source}(t) and P_{AC}(t) against the piston face with surface area A_{Piston}, drives the piston a distance 2R, resulting in a ΔX of 0 and the piston area A_{Vent}(x) is zero. As a result, the piston resistance is infinite, and flow is directed through the nares port to the lungs.

During the expiratory phase, t=0.9 to approximately t=2.1, Q_{Source}(t) is significantly reduced and flow come from the lungs back into the air chamber. At this point the combined force of the spring preload and pressure differential P_{AC}(t,) now being larger than force resulting from P_{Source}(t), the piston distance traveled is now back to zero and the vent location, ΔX is 2R, with the vent being completely open and R_{Vent}(t) is at a minimum. Exhaled air then exists the air chamber into the atmosphere.

When P_{AC}(t) drops below the desired PEEP level due to the reduce expiratory flow, approximately 5 CM H₂O, additional flow will be required from the Intelligent Gas Source in order to maintain the desired PEEP pressure. With P_{Source}(t) now being greater than P_{AC}(t) due to increased Q_{Source}(t), the piston is driven to a third location where ΔX is approximately ⅔R. At this point in time, the vent area and resistance are reduced so that a source gas flow of approximately 10 L/min will maintain an air chamber pressure P_{AC}(t) of 5 CM H2O. The entire cycle is then repeated.

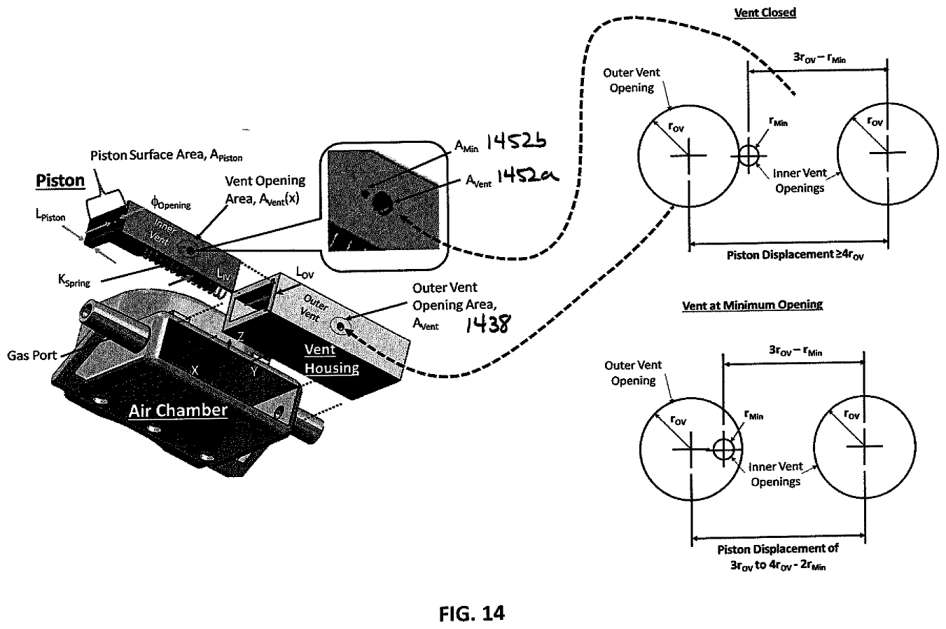

Another variable vent resistance configuration/embodiment is shown in FIG. 14. The Piston vent includes a large opening equivalent to the “Star” area and a small opening equivalent to the Minimum area illustrated in FIG. 14. The benefit of this design over the Star and Binary design is that flow control required to position the piston has a much looser tolerance than the other designs. Positioning for this design can be of the order of +/−1 mm where the star design positioning tolerance is half of that. The flow resistance for the three piston positions in X are outlined in Equations 22-26 as follows:

Diameter of Outer Vent, DOV

Large diameter of Inner Vent, D_{OV}, is equal to that of the outer vent

Small Diameter of Inner Vent, D_{Min}

Piston displacement when vent is in closed position:

Piston Displacement ≥4r_{OV}

R_{Vent}(4r_{OV)=∞} (22)

Piston Displacement when Small Inner Vent is within radius of outer vent:

Length of Inner Vent, L_{IV}

Piston Displacement equals 3r_{OV}−(4r_{OV}−2r_{Min})

$${D}_{Hydraulic} = Hydraulic Diameter$$

$$= {D}_{Min} for a circular tube$$

$${R}_{Min} = 8 {fL}_{Piston} ρ / {π}^{2} {D}_{Hydraulic}^{5} \qquad ( 23 )$$

$${P}_{AC} ( t ) - {P}_{Atmosphere} = {{Q}_{Vent} ( t )}^{2} {R}_{Min} \qquad ( 24 )$$

Vent is open, Large diameter inner vent opening is concentric with outer vent opening:

Piston displacement=0

Length of Outer Vent, L_{OV}

$${D}_{Hydraulic} = Hydraulic Diameter$$

$$= {D}_{OV} for a circular tube$$

$${R}_{OV} = 8 {fL}_{Piston} ρ / {π}^{2} {D}_{Hydraulic}^{5} \qquad ( 25 )$$

$${P}_{AC} ( t ) - {P}_{Atmosphere} = {{Q}_{Vent} ( t )}^{2} {R}_{OV} \qquad ( 26 )$$

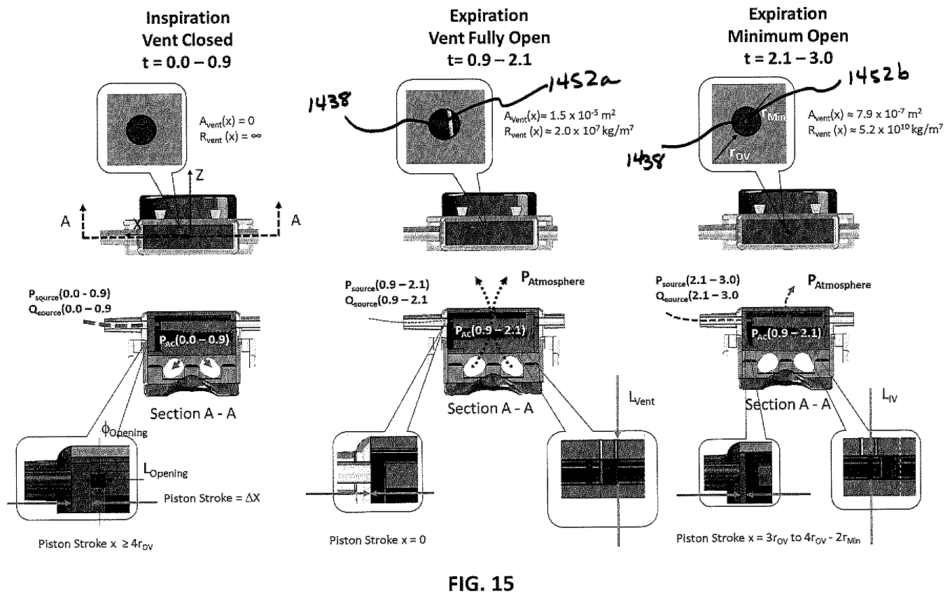

FIG. 15 illustrates operation of the embodiment of FIG. 14 during the breathing cycle. FIG. 15 shows the Piston position along the X axis, thus the associated vent area, A_{Vent}(x), and resistance, R_{Vent}(x), for the Inspiratory and Expiratory cycle as a function of time in seconds. During the Inspiratory phase of the cycle, t=0 to t=0.9 seconds, the Intelligent Gas Source provides the required flow, Q_{Source}(t), thorough the gas port and the resulting force due to the pressure difference between the P_{Source}(t) and P_{AC}(t) against the piston face with surface area A_{Piston}, drives the piston a distance x≥4r_{OV}, resulting in the piston area A_{Vent}(x) of zero. As a result, the piston resistance is infinite, and flow is directed through the nares port to the lungs. During the expiratory phase, t=0.9 to approximately t=2.1, Q_{Source}(t) is significantly reduced and flow come from the lungs back into the air chamber. At this point the combined force of the spring preload and pressure differential P_{AC}(t,) now being larger than force resulting from P_{Source}(t), the piston distance traveled is now x=0, with the vent being completely open and R_{Vent}(t) is at a minimum. Exhaled air then exists the air chamber into the atmosphere.

When P_{AC}(t) drops below the desired PEEP level due to the reduce expiratory flow, approximately 5 CM H₂O, additional flow will be required from the Intelligent Gas Source in order to maintain the desired PEEP pressure. With P_{Source}(t) now being greater than P_{AC}(t) due to increased Q_{Source}(t), the piston is driven to a third location where x=3r_{OV} to 4r_{OV}−2r_{Min}. At this point in time, the vent area and resistance are reduced so that a source gas flow of approximately 10 L/min will maintain an air chamber pressure P_{AC}(t) of 5 CM H₂O. The entire cycle is then repeated.

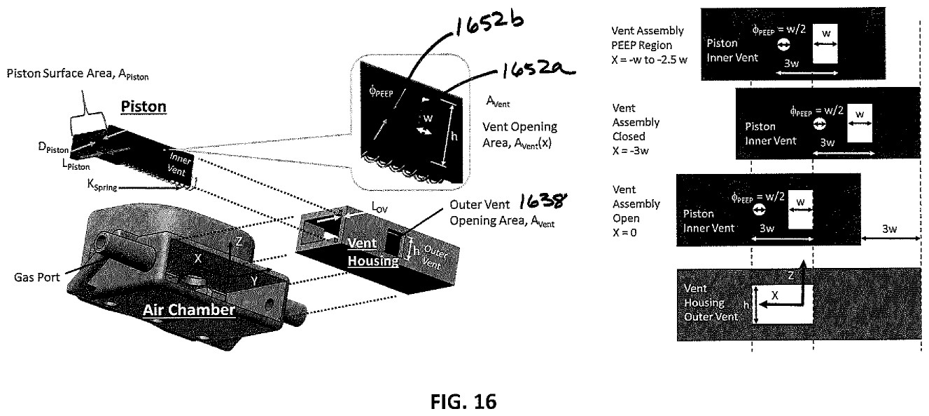

Another variable vent resistance configuration/embodiment is shown FIG. 16. The Piston vent includes a large rectangular opening equivalent to the “Star” area and a small circular opening equivalent to the Minimum area illustrated in FIG. 12 [symbol] . The benefit of this design over the Star and Binary design is that flow control required to position the piston has a much looser tolerance than the other designs. Positioning for this design can be of the order of +/−1 mm where the star design positioning tolerance is half of that. The flow resistance for the three piston positions in X are outlined in Equations 27-32 as follows:

Height of Outer Rectangular Vent, h

Height of Inner Rectangular Vent is equal to that of the outer vent

Width of Inner Rectangular Vent, w

Small Diameter of Inner Vent to achieve PEEP, D_{PEEP}

Piston displacement in x when vent is in closed position:

Piston Displacement=−3W

R_{Vent}(−3w)=∞ (27)

Piston Displacement when Small Inner Vent is within rectangular opening of outer vent:

Length of Inner Vent, L_{IV}

Piston Displacement in x equals −w to −2.5w

$${D}_{Hydraulic} = Hydraulic Diameter$$

$$= {D}_{PEEP} for a circular tube$$

$${R}_{PEEP} = 8 {fL}_{IV} ρ / {π}^{2} {D}_{Hydraulic}^{5} \qquad ( 28 )$$

$${P}_{AC} ( t ) - {P}_{Atmosphere} = {{Q}_{Vent} ( t )}^{2} {R}_{PEEP} \qquad ( 29 )$$

Vent is open, Large rectangular inner vent opening is aligned with outer vent opening:

$$Piston displacement = 0$$

$${L}_{IV} = Inner vent opening length$$

$${R}_{iVent} = Vent flow resistance$$

$${D}_{Hydraulic} = Hydraulic Diameter \qquad ( 30 )$$

$$= 2 wh ( w + h ) for a rectangular tube$$

$${R}_{Vent} = {R}_{Vent} = {fL}_{Vent} ρ ( w + h ) / 4 {( wh )}^{3} \qquad ( 31 )$$

Note that the hydraulic diameter is actually somewhat greater than what is called out in Equation 26 for the PEEP opening is also within the opening of the Outer Vent rectangle.

P_{AC}(t)−P_{Atmosphere}=Q_{Vent}(t)²R_{Vent} (32)

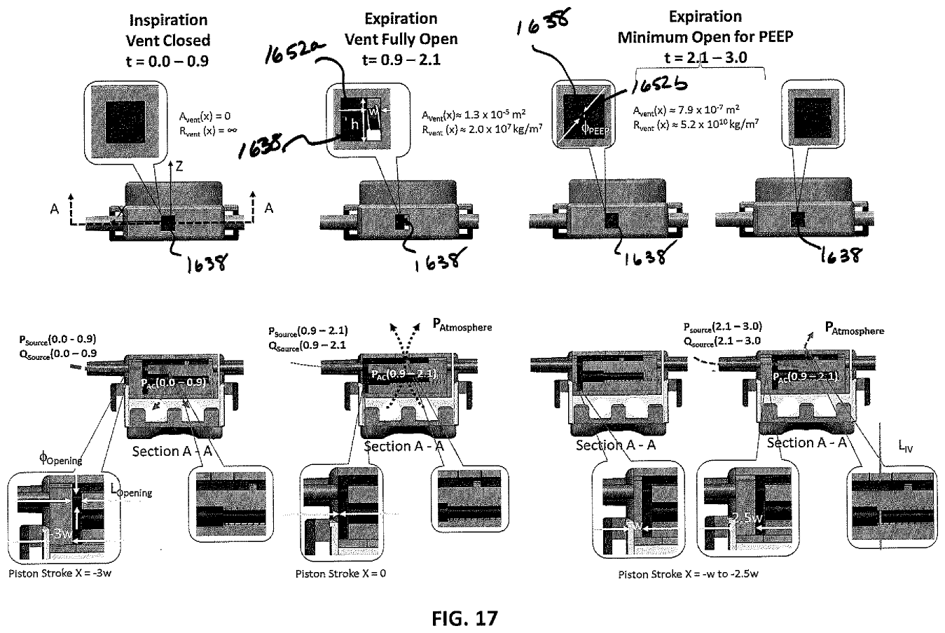

FIG. 17 illustrates operation of the embodiment of FIG. 16 during the breathing cycle. FIG. 17 shows the Piston position along the X axis, thus the associated vent area, A_{Vent}(x), and resistance, R_{Vent}(x), for the Inspiratory and Expiratory cycle as a function of time in seconds. During the Inspiratory phase of the cycle, t=0 to t=0.9 seconds, the Intelligent Gas Source provides the required flow, Q_{Source}(t), thorough the gas port and the resulting force due to the pressure difference between the P_{Source}(t) and P_{AC}(t) against the piston face with surface area A_{Piston}, drives the piston a distance −3w along the X axis, resulting in the piston area A_{Vent}(x) of zero. As a result, the piston resistance is infinite, and flow is directed through the nares port to the lungs.

During the expiratory phase, t=0.9 to approximately t=2.1, Q_{Source}(t) is significantly reduced and flow come from the lungs back into the air chamber. At this point the combined force of the spring preload and pressure differential P_{AC}(t,) now being larger than force resulting from P_{Source}(t), the piston distance traveled is now x=0, with the vent being completely open and R_{Vent}(t) is at a minimum. Exhaled air then exists the air chamber into the atmosphere.

When P_{AC}(t) drops below the desired PEEP level due to the reduce expiratory flow, approximately 5 CM H₂O, additional flow will be required from the Intelligent Gas Source in order to maintain the desired PEEP pressure. With P_{Source}(t) now being greater than P_{AC}(t) due to increased Q_{Source}(t), the piston is driven to a third location where x=−w to −2.5w. At this point in time, the vent area and resistance are reduced so that a source gas flow of approximately 10 L/min will maintain an air chamber pressure P_{AC}(t) of 5 CM H₂O. The entire cycle is then repeated.

Intelligent Gas Source

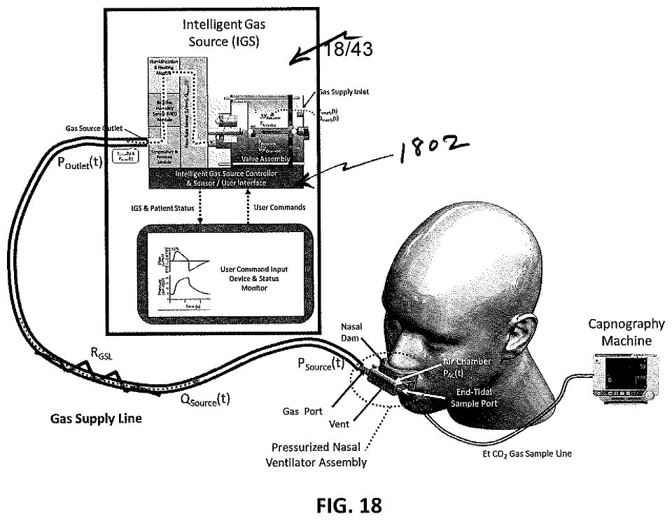

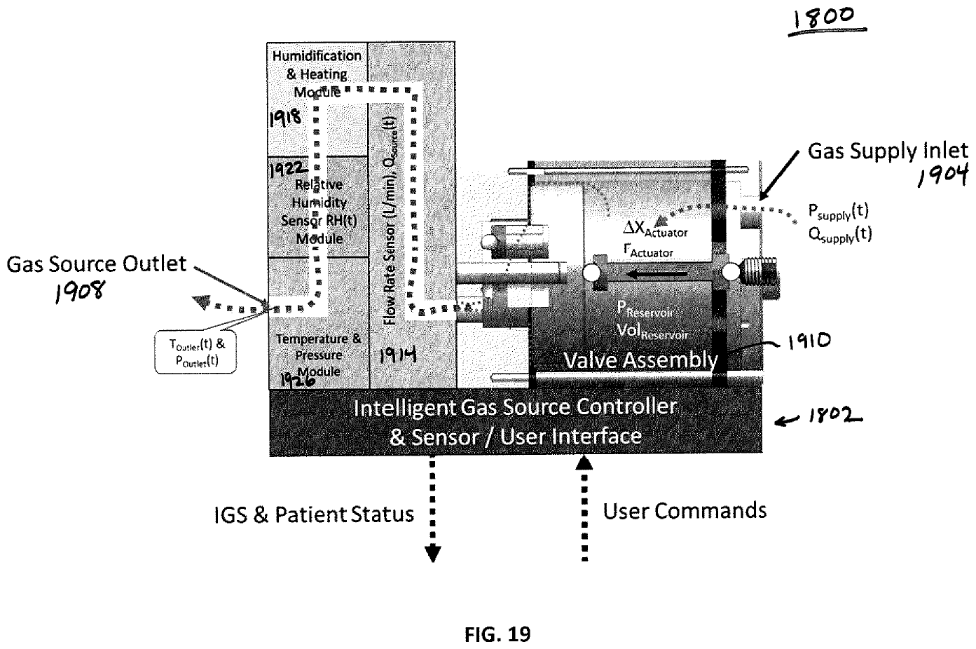

Further contemplated herein is an intelligent gas source for use with the previously described nasal ventilator assembly or other nasal ventilator assembly devices. FIG. 18 illustrates an intelligent gas source as use with a disclosed nasal ventilator apparatus is contemplated. As one of skill in the art would appreciate, the intelligent gas source may be used in other respiratory apparatus configurations within the scope of the present disclosure. The principles of the intelligent gas source will be described with respect to FIG. 19, which illustrates components of the intelligent gas source.

Referring to FIG. 18, provided is a respiratory ventilator that is smaller than current ventilator systems, with, for example, 1/10 to 1/100 the current volume and mass. Given the minimum part count and size, it is also anticipated to be less expensive to manufacture. The system disclosed in FIG. 18 includes an Intelligent Gas Source (IGS) 1800 that supplies the gas to the Pressurized Nasal Ventilator previously described. The IGS is responsible for supplying gas at the required flow rate and pressure that supports the desired respiratory cycle. A representative inhalation and exhalation cycle (flow and pressure provided by the ventilator) for a patient-ventilator interface during noninvasive ventilation is shown in FIG. 1. Inspiration of gas into the patient's lungs occurs when the flow rate as measured in L/min is positive while expiration occurs when the flow rate as measured in L/min is negative. Note that in this example, a minimum pressure of nominally 5 CM H₂O is maintained in order to provide Positive Expiratory End Pressure (PEEP). PEEP is a therapy provided in order to avoid passive emptying of the lung.

As can be seen in FIGS. 18 and 19, the intelligent gas source 1800 is a controller/user interface 1802, which will be described further herein. FIG. 19 illustrates components of the intelligent gas source 1800 that includes a gas supply inlet 1904 and a gas supply outlet 1908, which connects to a gas port 208 of a nasal respiratory apparatus as previously described or as part of another patient ventilator system. As can be seen in FIGS. 18 and 19, the intelligent gas source 1800 includes a valve assembly 1910 to control source gas flow by varying flow resistance, R_{Valve}(δ) a flow rate sensor 1914, humidification and heat module 1918, a relative humidity sensor module 1922, and a temperature and pressure source module 1926. Operation of each of these components will be described later. The intelligent gas source controller 1802 takes as inputs user commands and outputs status of the intelligent gas source 1800. Patient status may also be monitored.

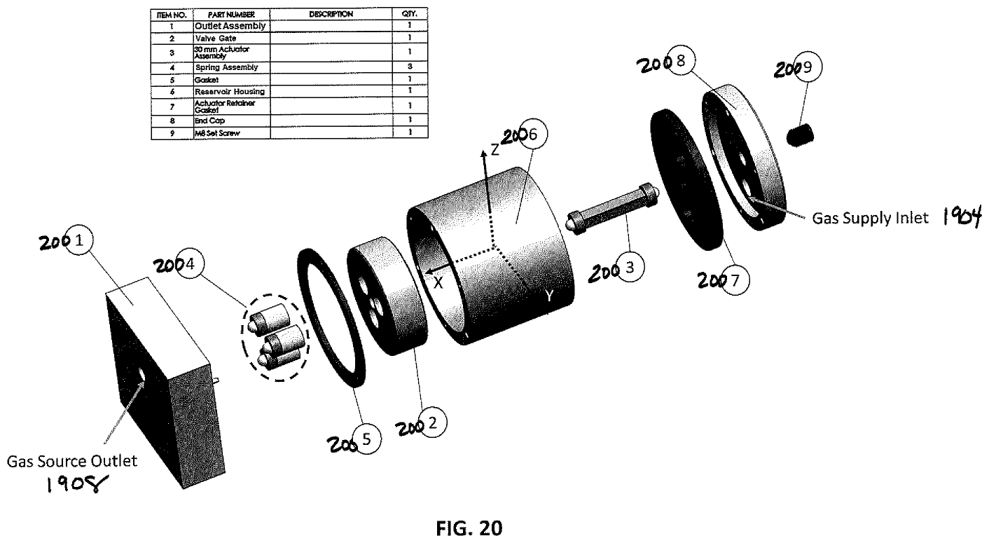

Details of an embodiment of the valve assembly 1910 are described with reference to FIG. 20. The valve assembly 1910 includes an outlet assembly 2001, a valve gate assembly 2002, an actuator assembly 2003, a spring assembly 2004, a gasket 2005, a reservoir housing 2006, an actuator retainer gasket 2007, an end cap 2008, and a screw set 2009.

The Outlet Assembly is the source low pressure side of the Valve Assembly. The Outlet Assembly is the source low pressure side of the Valve Assembly. Flow is controlled by the size of the gap along the X axis between the Y-Z face on the +X axis side of Valve gate and the Y-Z face on the −X axis side of the Outlet assembly. Gas flows annularly in the Y-Z plane towards the X axis between these two surfaces. The outlet can interface with multiple gas interface circuits for patients including but not limited to a standard 15 mm or 22 mm circuit or another gas supply line.

The Valve Gate controls the source flow rate, Q_{Source}(t) based on its position along the X axis. The distance between the Valve Gate Face on the positive X axis of the Gate with surface area A_{Gate}, and the Outlet Assembly Face, δ, determines the flow resistance by creating a resistance channel between the outlet assembly surface and the valve gate Y-Z face. Reservoir pressure, P_{Reservoir}, provides a force in the +X direction, F_{P}, where F_{P}=P_{Reservoir} A_{Gate}.

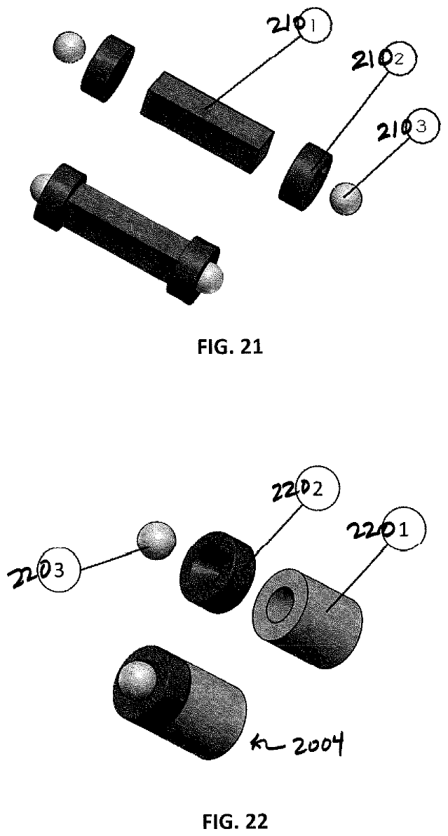

An embodiment of the Actuator Assembly 2003 is illustrated in FIG. 21and includes a P2T actuator 2101, an actuator bearing cup 2102 and a ball bearings 2103. The actuator assembly drives the Valve Gate along the X axis of the valve slide surface setting its position along the X axis. The assembly is a linear actuator that could be an electro-strictive material such as PZT or PMN, a magneto-strictive material, a screw drive or a voice coil drive or other linear drive mechanism. Its length and the resulting gate position is controlled under closed loop control based on the desired source flow rate, Q_{Source}(t) or flow source pressure, P_{Source}(t). It could also be driven under open loop control.

An embodiment of the spring assembly 2004 is illustrated in FIG. 22 and includes a spring 2201, an actuator bearing cup 2202 and a ball bearing 2203. A preload force in the negative X direction is applied to the Valve Gate and Actuator Assembly by the spring assembly 2004.

Gasket 2005 provides seal between the Outlet Assembly 2001 and Reservoir Housing 2006.

The Reservoir Housing 2006 contains the high-pressure side of the Valve Assembly. It acts as a gas reservoir, allowing the system to instantaneously supply a higher rate of gas flow than the average provided by the supply system, Q_{Supply}. The total volume of gas contained in the Reservoir Housing is governed by the Ideal gas law where n_{Reservoir}=P_{Reservoir}V_{Reservoir}/RT_{Reservoir}. n is the number of moles of gas, R is the universal gas constant and T_{Reservoir} is the reservoir gas temperature.

The actuator retainer 2007 gasket maintains a gas-tight seal between the Y-Z surfaces of the Reservoir Housing and the End Cap. It also positions the actuator assembly while configuring the Valve Assembly.

The End Cap 2008 encloses Y-Z face of the Valve Assembly. This cover creates a gas-tight seal between the Reservoir Housing and the atmosphere. The gas supply inlet is shown on the negative X axis face. The End Cap can interface with a standard hospital O2 source or with any gas source or compressor.

The set Screw 2009 drives the actuator assembly and valve gate in the X direction, setting both the spring assembly preload force and the Initial position of the Valve Gate along the X axis.

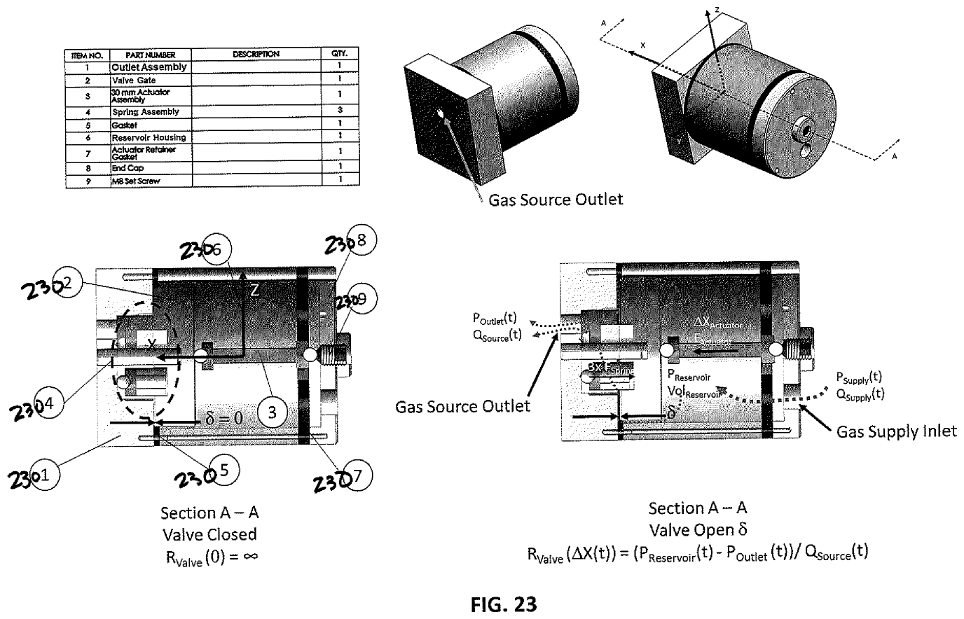

A cross-sectional view of the Valve Assembly is shown in FIG. 23. Section A-A on the left of the figure shows the valve in a closed position, δ=0, and the valve flow resistance, R_{Valve}(0) infinite. A cross sectional view on the right shows the valve assembly with the gate moved a distance δ, in the negative direction along the X axis. As a result the valve resistance is no longer infinite and gas flows from the reservoir to the Gas Source outlet as shown.

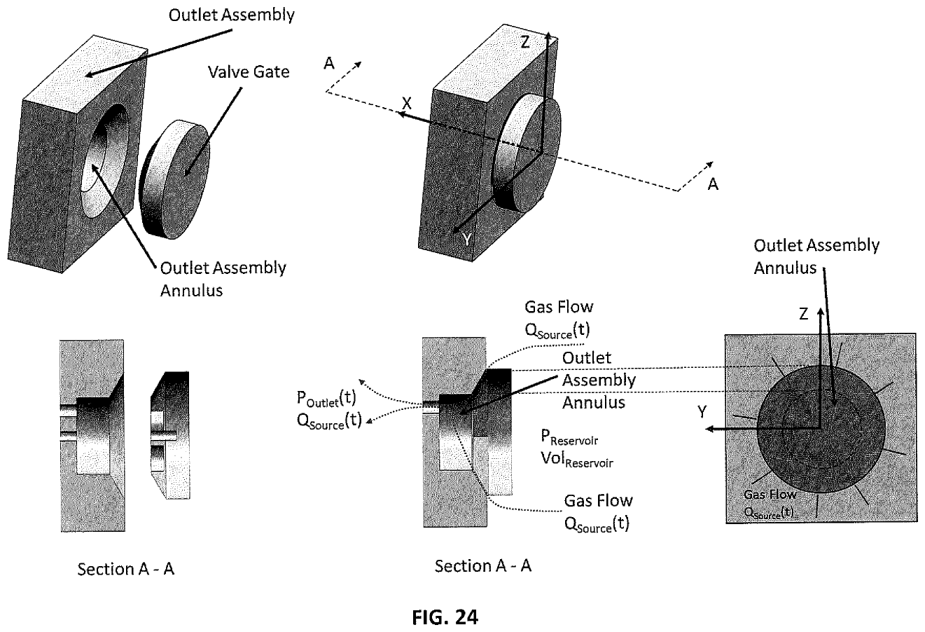

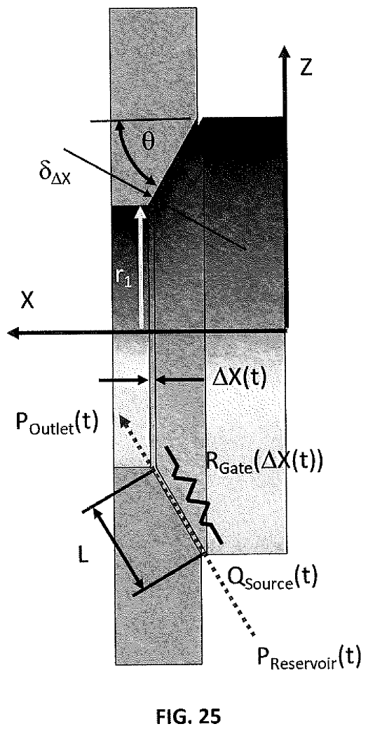

A general representation for the Outlet Assembly and Valve Gate are shown in FIGS. 24 and 25, where gas flow Q_{Source}(t) travels from the Reservoir with pressure P_{Reservoir}(t) around the perimeter of the Valve Gate, along the gap of length L into the annulus of the Outlet Assembly, with pressure P_{Outlet}(t). An approximation for the flow relationship can be made by assuming laminar flow through a set of parallel plates. In this case, through the gap between the Valve Gate and Outlet Assembly.

The valve flow resistance, R_{Valve}(ΔX(t)) can be modeled as a pneumatic circuit illustrated in FIG. 25. Source flow, Q_{Source}(t), is governed by Equation 4 where:

Reservoir Pressure, P_{Reservoir}(t)

Outlet Pressure, P_{Outlet}(t)

Source flow rate, Q_{Source}(t)

$$µ = Gas Viscosity$$

$$θ = Cone Angle$$

$$L = Radial Length of Overlap$$

$$Δ X ( t ) = Displacement of shaft along X Axis as a function of time t$$

$${δ}_{Δ X} ( t ) = Annulus gap ( Δ X ) \qquad ( 1 )$$

$$= Δ X ( t ) sin ( θ )$$

$${r}_{1} = Annulus inner Radius$$

$${A}_{AnnulusGap} = 2 π {r}_{1} {δ}_{Δ X} ( t ) = 2 π {r}_{1} Δ X ( t ) sin ( θ ) \qquad ( 2 )$$

$${R}_{Valve} ( Δ X ( t ) ) = 12 µ L / 2 π {{r}_{1} ( Δ X ( t ) sin ( θ ) )}^{3} \qquad ( 3 )$$

$$( {P}_{reservoir} ( t ) - {P}_{Outlet} ( t ) ) = {Q}_{Source} ( t ) {R}_{Valve} ( Δ X ( t ) ) \qquad ( 4 )$$

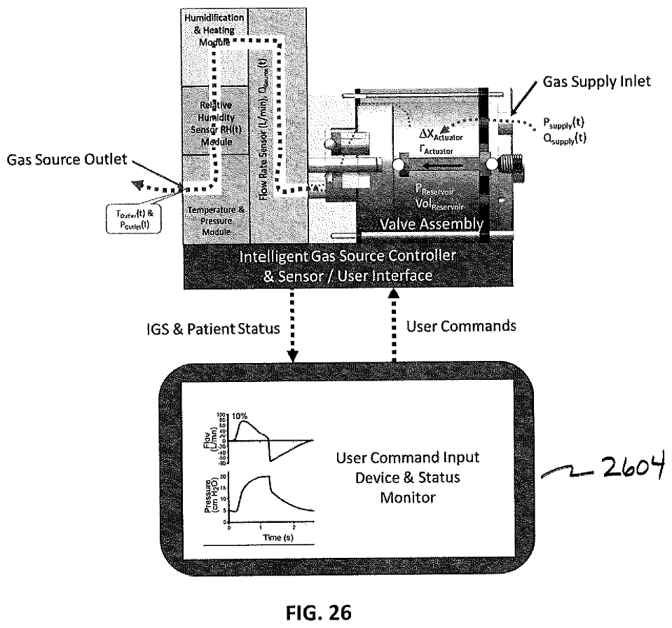

FIG. 26 illustrates principles of the intelligent gas source in interface with a user command input device and status monitor 2604. Referring to FIG. 26, The valve assembly controls source gas flow by varying flow resistance, R_{Valve}(δ). This is accomplished by changing the actuator length, ΔX, that result in moving the Gate valve along the X axis in corresponding gap between the valve gate and valve housing seal surface by δ. Reservoir pressure, P_{Reservoir}(t), is monitored and utilized by the Intelligent Gas Source Controller & Sensor/User Interface to calculate the required ΔX command that controls Q_{Source}(t), as outlined by Equation 3, above.

[symbol]

A gas reservoir region in the valve housing may be required, for while the average Source flow rate, Q_{Source}(t) does not exceed the available supply flow rate, Q_{Supply}(t), but the peak flow rate for Q_{Source}(t) does. This difference is made up from gas stored in the reservoir with volume Vol_{Reservoir} and pressure P_{Reservoir}.

Gas flows through the Flow Rate sensor measuring source flow, Q_{Source}(t) that is a function of time, t. This flow measurement is utilized by the Intelligent Gas Source Controller & Sensor/User Interface to calculate the required ΔX command that controls Q_{Source}(t) as outlined by Equation 4 or 7.

The air flow may require humidification and or heating. This is accomplished by commands from the Intelligent Gas Source Controller to the Humidification and Heat Module. This module adds water vapor, adding humidity to the gas flow, by either heating and subsequent evaporation of water, piezo atomization of water or other methods of adding water to the gas flow. The gas can also be heated by this module as the gas flows through it to the Gas Source Outlet.

The Temperature and Pressure Source Module measures Gas Temperature, T(t). This temperature measurement is utilized by the Intelligent Gas Source Controller & Sensor/User Interface to calculate the heating command, T_{Command}(t), to the Humidification and Heat Module to control gas temperature.

The Temperature and Pressure Source Module also measures gas outlet pressure, P_{Outlet}(t). This pressure is utilized by the Intelligent Gas Source Controller & Sensor/User Interface to calculate the required ΔX command that controls Q_{Source}(t) as outlined by Equation 4, above, or Equation 7, below. The outlet of the Temperature and Pressure Module interfaces with a Gas Supply that can include a standard 15 mm or 22 mm gas line or other gas line that terminates with a Pressurized Nasal Ventilator or other patient respiratory device such as a mask or cannula.

Gas flows through the Relative Humidity Sensor measuring gas relative humidity, RH(t) that is a function of time, t. This measurement is utilized by the Intelligent Gas Source Controller to generate the desired RH command, RH_{Command}(t) as a function of time.

The Intelligent Gas Source Controller & Sensor/User Interface includes the sensor interface required for controlling the gas source flow rate, Q_{Source}(t), pressure, P_{Outlet}(t), temperature T(t) and relative humidity, RH(t). It generates the actuator command, ΔX(t), the temperature command T_{Command}(t) and the relative humidity command RH_{Command}(t). It also interfaces with the User Command Input Device & Status Monitor, receiving the user defined command set for gas source flow rate, Q_{Source}(t), pressure, P_{Outlet}(t), T(t) and RH(t). The Intelligent Gas Source Controller & Sensor/User Interface also provides sensor readings to the User Command Input Device & Status Monitor.

The User Command Input Device & Status Monitor allows the user to generate commands for gas source flow rate, Q_{Source}(t), pressure, P_{Outlet}(t), T(t) and RH(t). It also displays sensor readings. This device can be an I-Pad-like interface that communicates with the Pressurized Nasal Ventilator Assembly in a wired or wireless fashion.

The Gas supply line can be a standard 6 mm O₂ line. The Gas supply line can also be insulated in order to minimize gas heat loss when traveling from the Intelligent Gas Source to the Pressurized Nasal Ventilator Assembly. The gas supply line can also incorporate am electrical heating element in order to maintain gas temperature. The Gas Supply Line can also incorporate a power and data wire set to provide power to the Pressurized Nasal Ventilator Assembly and receive sensor data from the Pressurized Nasal Ventilator Assembly. Note the gas supply line has a known flow resistance, R_{GSL}. The pressure at the point of entry to the Pressurized Nasal Ventilator Gas Port, P_{Source}(t) can be calculated as a result of knowing Q_{Source}(t), P_{Outlet}(t) and R_{GSL} by the equation P_{Source}(t)=P_{Outlet}(t) Q_{Source}(t) R_{GSL}.

Additional sensors can provide input for controlling the intelligent Gas Source Assembly. These include but are not limited to air chamber pressure, P_{Chamber}(t), air chamber temperature, TAC, air chamber relative humidity, RH_{AC}, ETCO₂ and or O₂ measurements sampled from the Pressurized Nasal Ventilator Assembly air chamber, impedance-based devices that monitor respiratory rate and tidal volume through chest cavity motion such as systems created by the Respiratory Motion ExSpiron system. See http://www.respiratorymotion.com/exspiron1xi.

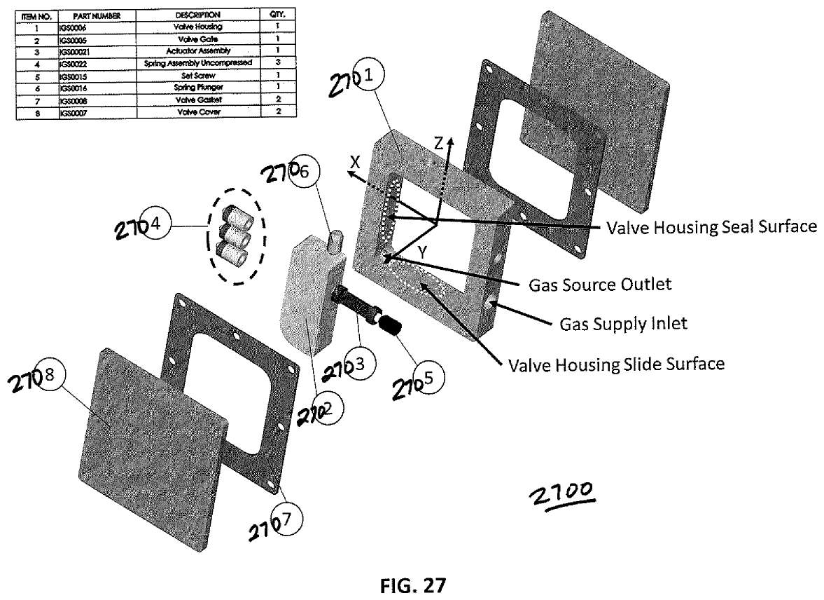

An alternate Valve Assembly 2700 is shown in FIG. 27. As illustrated in FIG. 27, the alternate valve assembly includes a valve housing 2701, a valve gate 2702, a actuator assembly 2703, a spring assembly 2704, a screw set 2705, a spring plunger 2706, a gasket 2707, and a valve cover 2708.

The Valve Housing contains the active elements of the Valve Assembly. The gas supply inlet is shown on the negative X axis face and the gas source outlet is on the positive X face. Additionally, the housing creates the gas reservoir. The Valve housing can interface with a standard hospital 02 source or with any gas source or compressor.

The Valve Gate controls the source flow rate, Q_{Source}(t) based on its position along the X axis. The face on the negative Z surface slides along the X axis on the Valve Housing Slide Surface. The distance between the Valve Gate Face on the positive X axis of the Gate and the Valve Housing Seal Surface, δ, determines the flow resistance by creating a resistance channel between the valve housing seal surface and the valve gate Y-Z face on the positive X axis.

The Actuator Assembly detailed in FIG. 21 drives the Valve Gate along the X axis of the valve slide surface setting its position along the X axis. The assembly is a linear actuator that could be an electro-strictive material such as PZT or PMN, a magneto-strictive material, a screw drive of a voice coil drive or other linear drive mechanism. It's length and the resulting gate position is controlled under closed loop control based on the desired source flow rate, Q_{Source}(t) or flow source pressure, P_{Source}(t). It could also be driven under open loop control.

With respect to the spring assembly 2704, a preload force in the negative X direction is applied to the Valve Gate and Actuator Assembly by the spring assembly. Detail of the assembly is provided in FIG. 22.

The set Screw 2705 drives the actuator assembly and valve gate in the X direction, setting both the spring assembly preload force and the Initial position of the Valve Gate along the X axis.

The Spring Plunger 2706 provides a preload to the Valve Gate in the negative Z direction. The intent is to continually maintain a gas-tight seal between the valve gate and valve housing slide surface.

The gasket 2707 maintains a substantially gas-tight seal between the X-Z surfaces of the Valve Housing and Valve Gate and the Valve Cover. It also greatest a gas-tight seal along the X-Z surface between the reservoir and the resistance channel.

The valve cover 2708 encloses X-Z face of the Valve Housing, one on the positive Y axis and one on the negative Y axis. These covers create a gas-tight seal between the Valve Housing and the atmosphere.

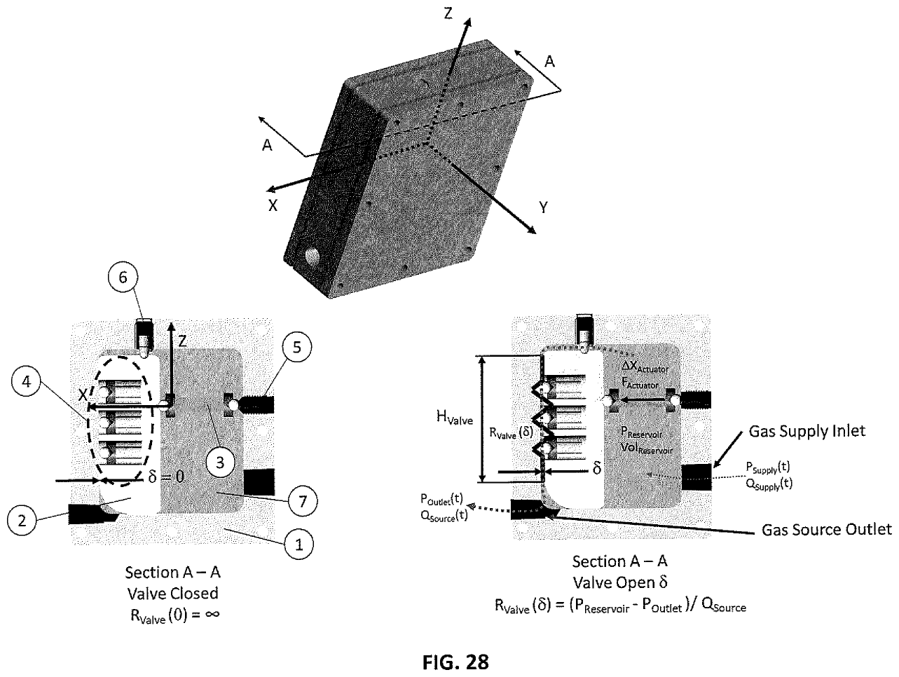

A cross-sectional view of the Valve Assembly is shown in FIG. 28. [symbol] . Section A-A on the left of the figure shows the valve in a closed position, δ=0, and the valve flow resistance, R_{Valve}(0) infinite. The valve elements previously described are also in the left-hand cross section. A cross sectional view on the right shows the valve assembly with the gate moved a distance δ, in the negative direction along the X axis. As a result the valve resistance is no longer infinite and gas flows from the reservoir to the Gas Source outlet as shown.

The valve flow resistance, R_{Valve}(δ) can be modeled as a pneumatic circuit illustrated in FIG. 28. The flow is modelled as flow between two parallel plates. Source flow, Q_{Source}(t), is governed by Equation 7 where:

Reservoir Pressure, P_{Reservoir}(t)

Outlet Pressure, P_{Outlet}(t)

Source flow rate, Q_{Source}(t)

Valve Height, H_{Valve}

Valve Depth along the Y axis, D_{Valve}

Valve Gate distance from valve housing sealing surface, δ

$${A}_{Resistance} ( δ ) = Resistance Channel Cross - sectional area \qquad ( 5 )$$

$$= {D}_{Valve} δ$$

Gas Dynamic Viscosity, μ (mass/(distance−time)

$${R}_{Valve} ( δ ) = Vent flow resistance through the resistance channel \qquad ( 6 )$$

$$= 12 µ {H}_{Valve} / {D}_{Valve} {δ}^{3}$$

Source Flow Rate can then be determined by the following relationship:

Q_{Source}(t)=(P_{Reservoir}(t)−P_{Outlet}(t)/R_{Valve}(δ) (7)

A gas reservoir region in the valve housing is required, for while the average Source flow rate, Q_{Source}(t) does not exceed the available supply flow rate, Q_{Supply}(t), but the peak flow rate for Q_{Source}(t) does. This difference is made up from gas stored in the reservoir.

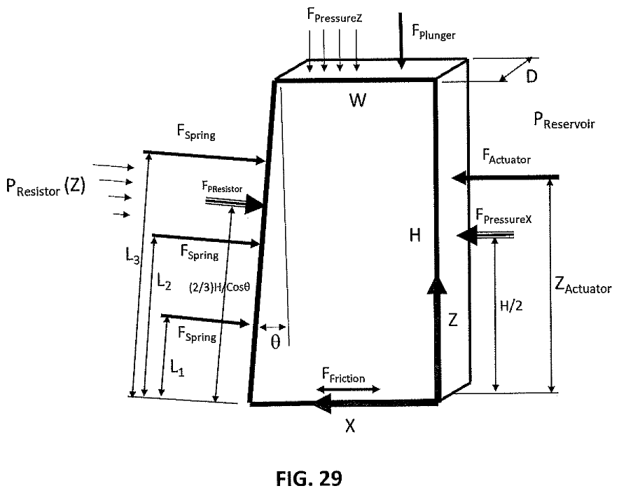

The force and moment balance for a generalized Valve Gate is illustrated in FIG. 29. Governing equations for both the force balance and moment balance are provided in Equations 8-16, below.

$${P}_{Reservoir} = Reservoir pressure$$

$$θ = Valve Gate Angle$$

$$W = Valve Gate Width$$

$$H = Valve Gate Height$$

$${H}_{Valve} = H / Cos θ \qquad ( 8 )$$

$${D}_{Valve} = Valve Gate Depth$$

$${L}_{1} , {L}_{2} \& {L}_{3} = Spring Distance$$

$${c}_{Friction} = Wedge Friction Coefficient$$

$${Z}_{Actuator} = Actuator Distance from Z = 0$$

$${P}_{Reservoir} = Reservoir gas pressure$$

$${F}_{PressureX} = force from chamber pressure in - X direction \qquad ( 9 )$$

$$= - {P}_{Reservoir} HD$$

$${F}_{PressureZ} = force from chamber pressure in - Z direction \qquad ( 10 )$$

$$= - {P}_{Reservoir} ( WD + H tan θ D / 2 )$$

$${F}_{Plunger} = Force from Spring Plunger in Z direction$$

$${P}_{Resistor} ( Z ) = Pressure along flow resistor wall \qquad ( 11 )$$

$$\approx {P}_{Reservoir} ( Z / H )$$

$${F}_{PResistor} = Force on Resistor wall from Pressure due to flow \qquad ( 12 )$$

$$= ( {P}_{Reservoir} / 2 ) {D}_{Valve} {H}_{Valve}$$

$${F}_{Spring} = Force provided by preload spring$$

$${F}_{Actuator} = Force provided by positioning actuator$$

Z Force Balance

$${F}_{Z} = {F}_{Plunger} + {F}_{PressureZ} + 3 {F}_{Spring} Sin θ \qquad ( 13 )$$

$${F}_{Friction} = Friction force in X direction \qquad ( 14 )$$

$$= {F}_{Z} {c}_{Friction}$$

X Force Balance

(3F_{Spring}+F_{PResistor})Cos θ=F_{PressureX}+F_{Actuator} (15)

Moment Balance About Y Axis

$${F}_{Spring} ( {L}_{1} + {L}_{2} + {L}_{3} ) Cos {θ}^{2} + {F}_{PResistor} ( ( 2 / 3 ) H / Cos θ ) Cos {θ}^{2} \qquad ( 16 )$$

$$= {F}_{PressureX} H / 2 + {F}_{Actuator} {Z}_{Actuator}$$

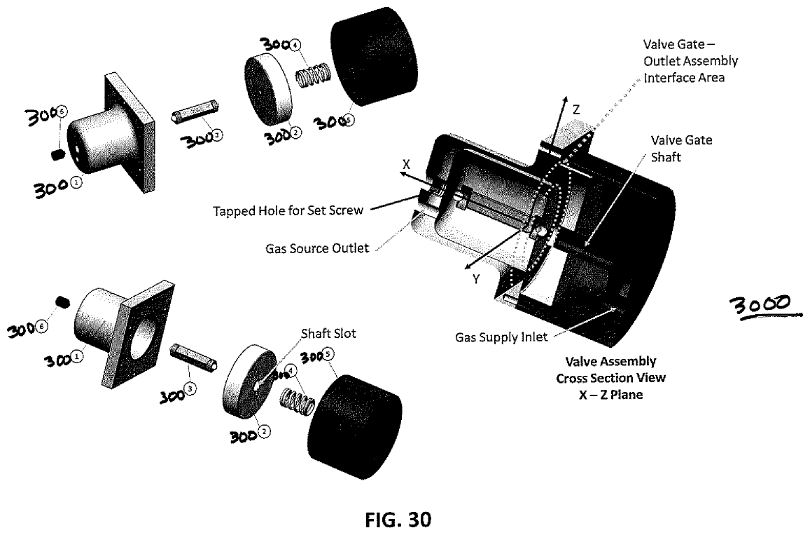

Another alternate Valve Assembly is shown in FIG. 30 with the actuator on the low-pressure side of the valve gate. Detail of the Valve Assembly is provided are provided below. There are several benefits of this design over the valve assembly with the actuator on the high pressure side of the valve gate. These include: (1) Reservoir pressure provides the preload required for the piezo actuator so that large preload springs are not required; (2) there is a lower part count, and (3) if the actuator fails mechanically or electrically, the Valve Gate will close. while a valve assembly with an actuator on the high pressure side may be desirable, it is possible that the valve gate would fail open, allowing high pressure flow through the system potentially hurting a patient, such that an alternative may be desired.

Referring again to FIG. 30, the alternate valve assembly 3000 includes an outlet assembly 3001, a valve gate 3002, an actuator assembly 3003, a spring assembly 3004, a reservoir housing 3005, and a set screw 3006. The Outlet Assembly is the source low pressure side of the Valve Assembly. Flow is controlled by the size of the gap along the X axis between the Y-Z face on the +X axis side of Valve gate and the Y-Z face on the −X axis side of the Outlet assembly. This interface area is highlighted in FIG. 30. Gas flows annularly in the Y-Z plane towards the X axis between these two surfaces. The Gas Source outlet can interface with multiple gas interface circuits for patients including but not limited to a standard 15 mm or 22 mm circuit or another gas supply line. There is also a tapped hole for the Set Screw and subsequent actuator position adjustment along the X axis on the Y-Z face of the +X axis.

Referring to the valve gate 3002, the Valve Gate Y-Z Surface on the +X axis faces the Outlet Assembly Y-Z surface on the −X axis. The Valve Gate 3002 controls the source flow rate, Q_{Source}(t) based on its position along the X axis and the resulting distance between these two surfaces. The distance between the Valve Gate Face on the positive X axis of the Gate with surface area A_{Gate}, and the Outlet Assembly Face, δ, determines the flow resistance by creating a resistance channel between the outlet assembly surface and the valve gate Y-Z face. Reservoir pressure, P_{Reservoir}, provides a force in the +X direction, F_{P}, where F_{P}=P_{Reservoir} A_{Gate}. The Valve Gate also has a slot with a circular cross section that interfaces with the Valve Gate Shaft. The overlap region between the Valve Gate and Outlet Assembly is illustrated in FIG. 30.

The Actuator Assembly 3003 detailed in FIG. 30 interfaces with the Valve Gate, positioning the Valve Gate along the X axis of the valve slide surface in the Y-Z plane, setting its position along the X axis. The assembly is a linear actuator that could be an electro-strictive material such as PZT or PMN, a magneto-strictive material, a screw drive or a voice coil drive or other linear drive mechanism. It's length and the resulting gate position is controlled under closed loop control based on the desired source flow rate, Q_{Source}(t) or flow source pressure, P_{Source}(t). I could also be driven under open loop control.

The spring assembly 3004 provides a preload force in the positive X direction is applied to the Valve Gate and Actuator Assembly by the spring assembly necessary only to assure the gate is in a closed position prior to the reservoir being pressurized so there is no leakage during pressurization. The bulk of the preload is provided by the Reservoir pressure when operational.

The Reservoir Housing 3005 contains the high-pressure side of the Valve Assembly. It acts as a gas reservoir, allowing the system to instantaneously supply a higher rate of gas flow than the average provided by the supply system, Q_{Supply}. The total volume of gas contained in the Reservoir Housing is governed by the Ideal gas law where n_{Reservoir}=P_{Reservoir}V_{Reservoir}/RT_{Reservoir}. n is the number of moles of gas, R is the universal gas constant and T_{Reservoir} is the reservoir gas temperature. The Reservoir Housing 3005 has a Gas Supply Inlet can interface with a standard hospital O2 source or with any gas source or compressor. The areservoir housing also contains a Valve Gate Shaft that is inserted into the gate, restraining it in the Y-Y plane but allowing for travel along the X axis.

The Set Screw 3006 resides in the Tapped Hole of the Outlet Assembly and interfaces with the actuator Assembly. Rotating the Screw drives the actuator assembly and valve gate in the X direction, setting the Initial position of the Valve Gate along the X axis.

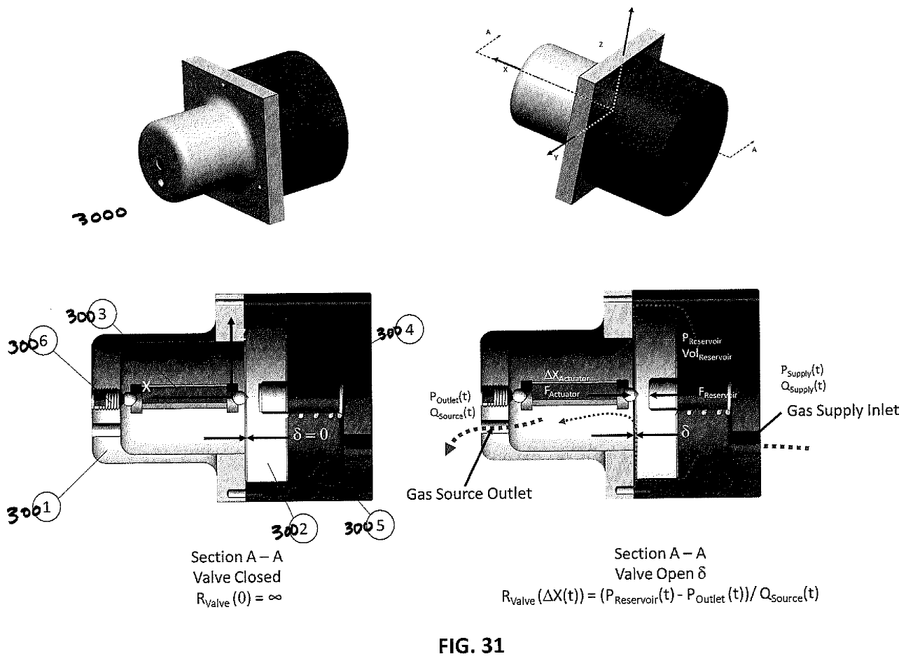

A cross-sectional view of the Valve Assembly with the actuator on the low-pressure side of the Valve Gate is shown in FIG. 31. Section A-A on the left of the figure shows the valve in a closed position, δ=0, and the valve flow resistance, R_{Valve}(0) infinite. The valve elements with callouts that were previously described are also in the left hand cross section. A cross sectional view on the right shows the valve assembly with the gate moved a distance δ, in the negative direction along the X axis. As a result, the valve resistance is no longer infinite and gas flows from the reservoir to the Gas Source outlet as shown.

A general representation for the Outlet Assembly and Valve Gate are shown in FIG. 24 where gas flow Q_{Source}(t) travels from the Reservoir with pressure P_{Reservoir}(t) around the perimeter of the Valve Gate, along the gap of length L into the annulus of the Outlet Assembly, with pressure P_{Outlet}(t). An approximation for the flow relationship can be made by assuming the flow through a set of parallel plates. In this case, through the gap between the Valve Gate and Outlet Assembly.

The valve flow resistance, R_{Valve}(ΔX(t)) can be modeled as a pneumatic circuit illustrated in FIG. 25. Source flow, Q_{Source}(t), is governed by Equation 7.

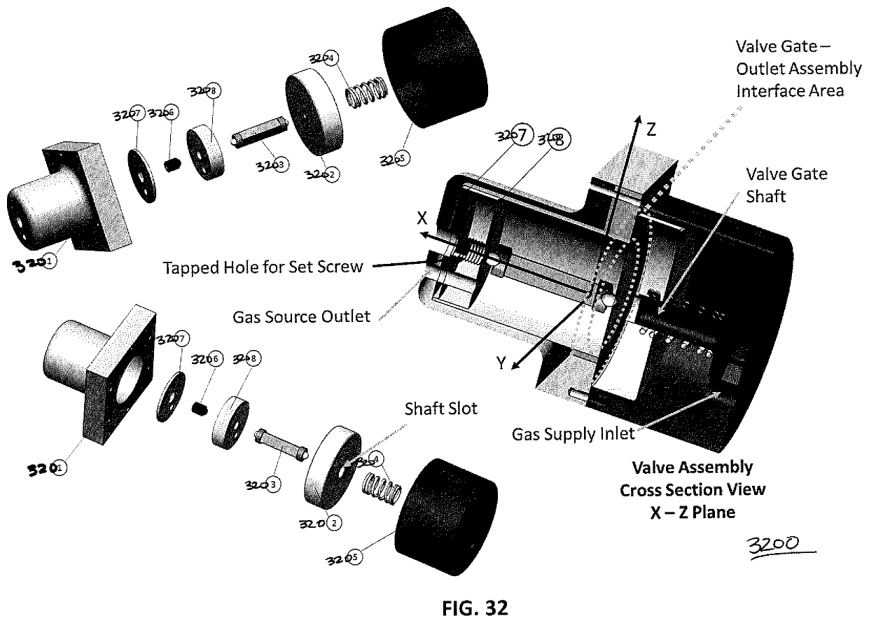

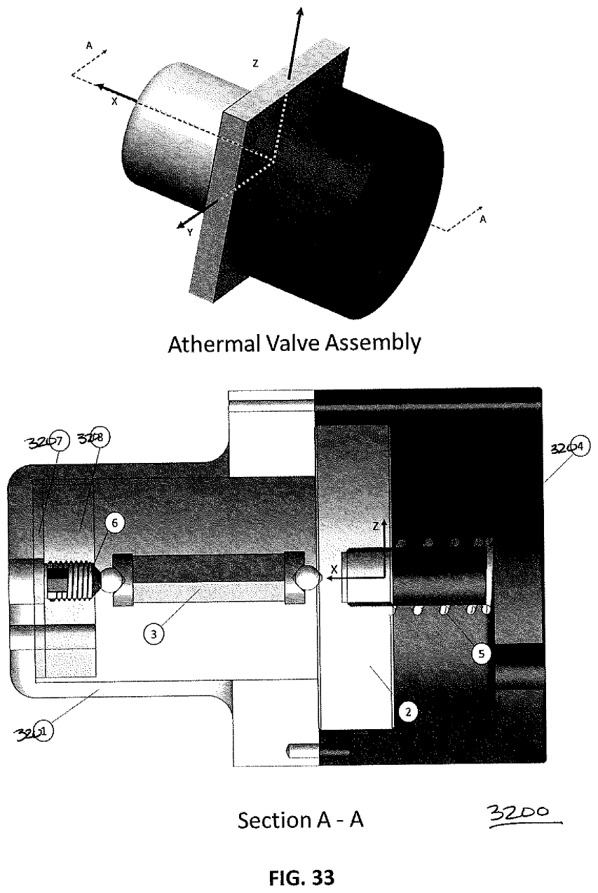

An example athermal Valve Assembly is shown in FIGS. 32 and 33, with the actuator on the low-pressure side of the valve gate. The example athermal valve assembly 3200 includes an outlet assembly 3201, a valve gate 3202, an actuator assembly 3203, a spring assembly 3204, a reservoir housing 3205, a set screw 3206, an athermal spacer 3207, and an actuator reaction plate 3208.

The Outlet Assembly is the source low pressure side of the Valve Assembly. Flow is controlled by the size of the gap along the X axis between the Y-Z face on the +X axis side of Valve gate and the Y-Z face on the −X axis side of the Outlet assembly. Gas flows annularly in the Y-Z plane towards the X axis between these two surfaces. This interface area is highlighted in FIG. 32. The Gas Source outlet can interface with multiple gas interface circuits for patients including but not limited to a standard 15 mm or 22 mm circuit or another gas supply line. There is a hole in the end of the Outlet Assembly on the Y-Z face that allows for access to the set screw in the Actuator Reaction Plate.

The Valve Gate Y-Z Surface on the +X axis faces the Outlet Assembly Y-Z surface on the −X axis. The Valve Gate controls the source flow rate, Q_{Source}(t) based on its position along the X axis and the resulting distance between these two surfaces. The distance between the Valve Gate Face on the positive X axis of the Gate with surface area A_{Gate}, and the Outlet Assembly Face, δ, determines the flow resistance by creating a resistance channel between the outlet assembly surface and the valve gate Y-Z face. Reservoir pressure, P_{Reservoir}, provides a force in the +X direction, F_{P}, where F_{P}=P_{Reservoir} A_{Gate}. The Valve Gate also has a slot with a circular cross section that interfaces with the Valve Gate Shaft. The overlap region between the Valve Gate and Outlet Assembly is illustrated in FIG. 32.

The Actuator Assembly detailed in FIG. 21 interfaces with the Valve Gate, positioning the Valve Gate along the X axis of the valve slide surface in the Y-Z plane, setting its position along the X axis. The assembly is a linear actuator that could be an electro-strictive material such as PZT or PMN, a magneto-strictive material, a screw drive or a voice coil drive or other linear drive mechanism. Its length and the resulting gate position is controlled under closed loop control based on the desired source flow rate, Q_{Source}(t) or flow source pressure, P_{Source}(t). It could also be driven under open loop control.

The spring assembly provides a preload force in the positive X direction is applied to the Valve Gate and Actuator Assembly by the spring assembly necessary only to assure the gate is in a closed position prior to the reservoir being pressurized so there is no leakage during pressurization. The bulk of the preload is provided by the Reservoir pressure when operational.

The Reservoir Housing contains the high-pressure side of the Valve Assembly. It acts as a gas reservoir, allowing the system to instantaneously supply a higher rate of gas flow than the average provided by the supply system, Q_{Supply}. The total volume of gas contained in the Reservoir Housing is governed by the Ideal gas law where n_{Reservoir}=P_{Reservoir}V_{Reservoir}/RT_{Reservoir}. n is the number of moles of gas, R is the universal gas constant and T_{Reservoir} is the reservoir gas temperature. The Reservoir Housing has a Gas Supply Inlet can interface with a standard hospital O2 source or with any gas source or compressor. The reservoir housing also contains a Valve Gate Shaft that is inserted into the gate, restraining it in the Y-Y plane but allowing for travel along the X-axis.

The Set Screw resides in the Tapped Hole of the Outlet Assembly and interfaces with the actuator Assembly. Rotating the Screw drives the actuator assembly and valve gate in the X direction, setting the Initial position of the Valve Gate along the X axis.

The Athermal Spacer attaches to the Y-Z face on the −X axis side of the Outlet Assembly end and the Y-Z face on the +X axis side of the Actuator Reaction Plate. The CTE and thickness of the spacer are chosen to compensate for the CTE difference between the Actuator Assembly and the Outlet Assembly. There is no contact between the Athermal Spacer surfaces perpendicular to the Y-Z plane and the Outlet Assembly.

The Actuator Reaction Plate attaches to the Y-Z face on the −X axis side of Athermal Spacer and has the same CTE as the Outlet Assembly. There is also a tapped hole for the Set Screw and subsequent actuator position adjustment along the X axis. There is no contact between the Actuator Reaction Plate surfaces perpendicular to the Y-Z plane and the Outlet Assembly.

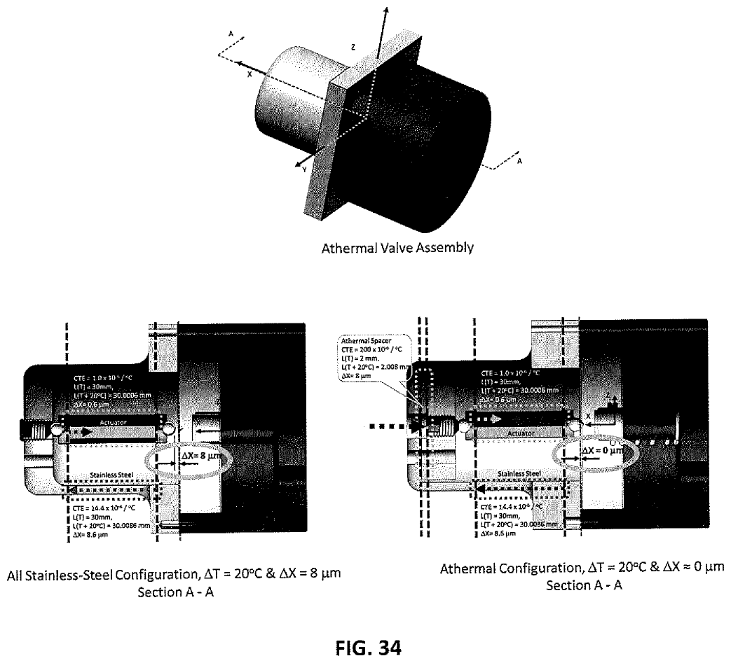

There are several benefits of this design over non-athermal valve assemblies, both with the actuator on the high-pressure side of the valve gate and with the actuator on the high pressure side of the valve gate including reducing sensitivity to changes in temperature is very important due to the factor of >14× difference in the coefficient of thermal expansion (CTE) between the actuator and Outlet Assembly. An illustration of the impact is shown in FIG. 34 where a representative CTE for the actuator is 10⁻⁶/° C., while the CTE for an Outlet Assembly made of stainless steel is 14.4×10⁻⁶/° C. A 20° C. increase in temperature over the temperature where the Valve Assembly was initially configured, would result in an 8 μm gap between the actuator and Valve Gate face. This is a significant distance, given the maximum actuator stroke anticipated for operation is nominally 45 μm. If the temperature dropped by 20° C., the Valve Gate would be driven from the Outlet Assembly interface surface by 8 μm, resulting in a constant leak in the valve.

An all stainless Valve Assembly configuration is illustrated on the left side of FIG. 34. During initial configuration of the Valve Assembly at a temperature of T_{Config}, the set screw is rotated until driving the actuator assembly to be in contact with the Y-Z surface on the +X axis of the Valve Gate. From this point, the actuator can increase in length, positioning the valve gate along the X axis from the initial closed position at X=0 to the fully open position at X=45 μm. The Set Screw is attached to the end of the Outlet Assembly.

If there is an increase in temperature, for this example 20° C., Outlet housing expands and the set screw moves with it in the +X direction from the Valve Gate. In a similar manner, the Actuator assembly also expands, but the motion is in the −X direction towards the Valve Gate. If the Outlet Housing and Actuator Assembly had the same CTE, the actuator would maintain contact with the Y-Z surface of the Valve Gate. Since there is a >14× difference in CTE, an 8 μm gap between the end of the Actuator Assembly and Valve gate occurs as outlined in Equation 18.

$${CTE}_{Actuator} = Coefficient of thermal expansion for the Actuator Assembly$$

$$= {10}^{- 6} / ° C .$$

$${L}_{Actuator} ( T ) = Actuator Length as a function of temperature , T$$

$$= 30 mm at Valve Assembly Configuration temperature {T}_{Config}$$

$$Δ T = Temperature Difference from the Valve Assembly configuration and current temperature$$

$$= 20 ° C .$$

$$Δ {L}_{Actuator} ( Δ T ) = Change in actuator length from the Valve Assembly configuration and current temperature \qquad ( 17 )$$

$$= {L}_{Actuator} ( {T}_{Config} ) Δ T {CTE}_{Actuator}$$

$$= 0.6 µm ( Positive Δ {L}_{Actuator} results in motion in - X direction )$$

$${CTE}_{Outlet} = Coefficient of thermal expansion for the outlet assembly$$

$$= 14.4 \times {10}^{- 6} / ° C .$$

$${L}_{Outlet} ( T ) = Outlet Assembly length corresponding to actuator length as a function of temperature , T$$

$$= 30 mm at Valve Assembly Configuration temperature {T}_{Config}$$

$$Δ {L}_{Outlet} ( Δ T ) = Change in Outlet Assembly length from the Valve Assembly configuration and current temperature \qquad ( 18 )$$

$$= {L}_{Outlet} ( {T}_{Config} ) Δ T {CTE}_{Outlet}$$

$$= 8.6 µm ( Positive Δ {L}_{Outlet} results in motion in + X direction )$$

Given the positive 20° C. temperature differential, outlet housing results in motion in the +X direction and the Actuator Assembly motion results in motion in the −X direction. Subtracting Equation 17 from 18 results in an 8 μm gap between the end of the Actuator Assembly and the Valve Gate face. A negative 20 ° C. temperature difference from the configuration temperature results in the actuator driving the valve gate 8 μm in the −X direction. This results in a gap between the Outlet Assembly and Valve Gate.

The Athermal design compensates for the CTE difference by placing a very high CTE Athermal Spacer in the X load path as shown by the Athermal Valve Assembly on the right side of [symbol] . The CTE and thickness along the X axis are chosen to compensate for the 14× difference in CTE between the Actuator Assembly and Outlet Assembly. Note that the side of the spacer perpendicular to the Y-Z plane are not in contact with the Outlet Assembly. It is assumed that all other material in the reaction path of the actuator has the same CTE as the Outlet Assembly. The following outlines the Athermal Spacer expansion as a function of temperature:

$${CTE}_{Athermal} = Coefficient of thermal expansion for the Athermal Spacer$$

$$= 200 \times {10}^{- 6} / ° C .$$

$${L}_{Athermal} ( T ) = Athermal Spacer length along the X axis as a function of temperature , T$$

$$Δ {L}_{Athermal} ( Δ T ) = Change in Athermal Spacer length from the Valve Assembly configuration and current temperature \qquad ( 19 )$$

$$= {L}_{Athermal} ( {T}_{Config} ) Δ T {CTE}_{Athermal}$$

An increase in temperature relative to the configuration temperature results in an expansion of the Athermal spacer with resulting motion in the −X direction, as with the Actuator Assembly. The objective is to have the Athermal Spacer and Actuator Assembly thermal expansion equal that of the Outlet Assembly, thus eliminating a gap between the actuator and the Valve Gate as outlined in Equation 20:

ΔL_{Outlet}(ΔT)=ΔL_{Athermal}(ΔT)+ΔL_{Actuator}(ΔT) (20)

L_{Outlet}(T_{Config})ΔT CTE_{Outle}=L_{Athermal}(T_{Config})ΔT CTE_{Athermal}+L_{Actuator}(T_{Config})ΔT CTE_{Actuator}

Restating Equation 16 results in the following solution for L_{Athermal}(T_{Config}):

$${L}_{Athermal} ( {T}_{Config} ) \qquad ( 21 )$$

$$= ( {L}_{Outlet} ( {T}_{Config} ) {CTE}_{Outle} - {L}_{Actuator} ( {T}_{Config} ) {CTE}_{Actuator} ) / {CTE}_{Athermal}$$

$$= 2 mm$$

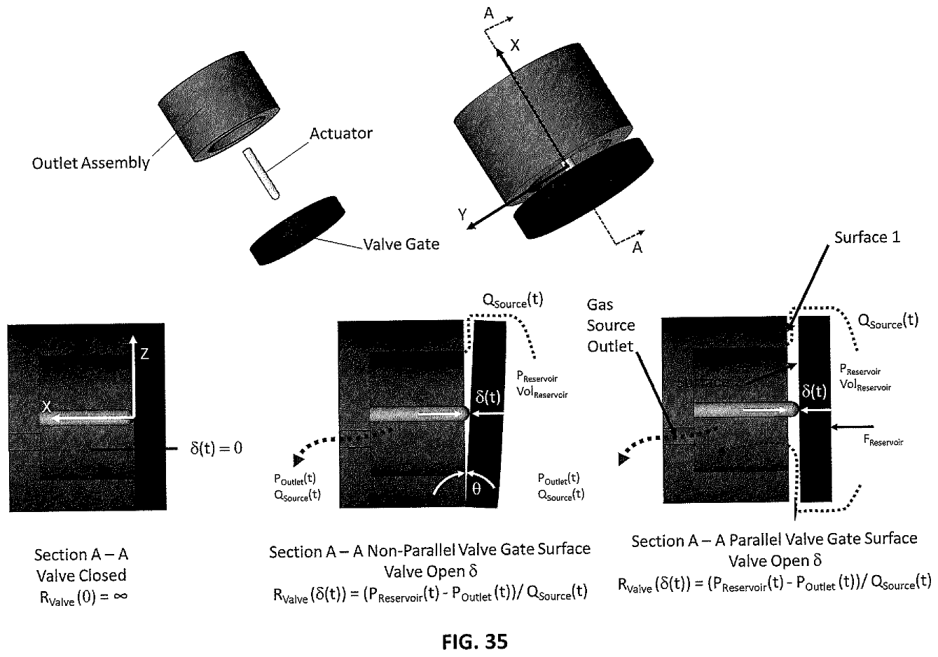

A simplified illustration of the valve assembly shown in FIG. 32 is provided in FIG. 35 showing the Outlet Assembly, the Valve Gate and the Actuator. The actuator extends and contracts axially and is nominally parallel to Surface 1 of the Outlet Assembly and Surface 2 of the Valve Gate. When extended, a gap of length d(t) between these two surfaces results and gas flows at a rate Q_{Source}(t) from the reservoir at pressure P_{Reservoir}between surface 1 of the outlet assembly and surface 2 of the valve gate into the Outlet assembly and then out the Gas Source Outlet as shown by the Section A-A for the parallel and non-parallel valve gate configurations. Q_{Source}(t) is proportional to the gap, δ(t) between surface 1 and surface 2. One end of the actuator is attached to the outlet assembly with surface 1, and the other end is attached to surface 2 of the valve gate. The actuator extension, δ(t) is anticipated to range from 0 μm to 100 μm. It is anticipated that due to machining tolerances, surface and surface 2 will be non-parallel. In the non-parallel configuration shown in FIG. 35, as the gate begins to close the surface will initially contact at a point where the average gap distance is then δ(t). As long as the valve gate is not constrained from rotating about either the X or Y axis, the average surface distance will continue to be reduced as d(t) is reduced, until it is completely closed when d(t)=0.

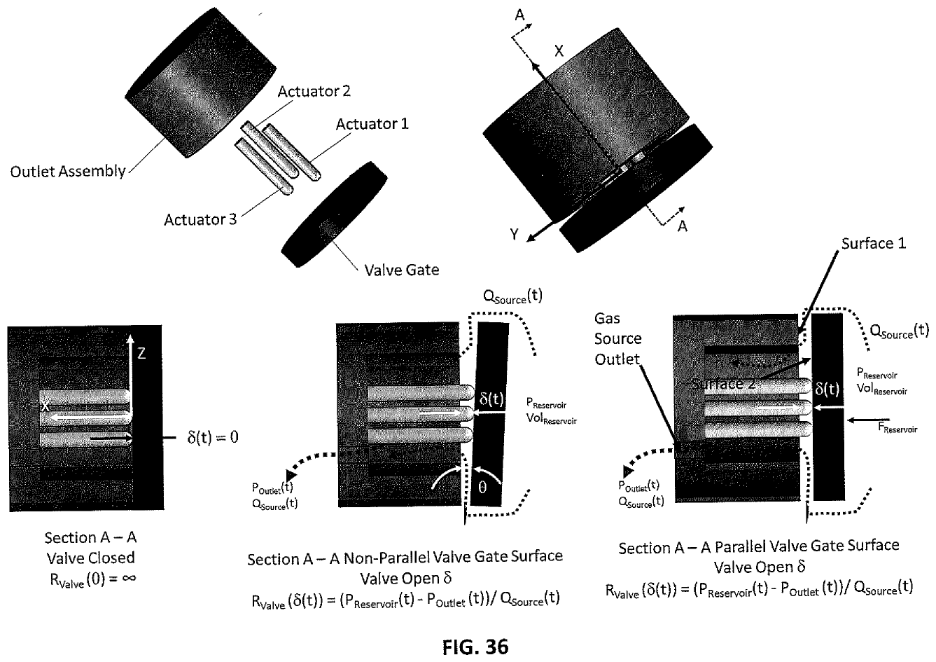

A simplified illustration showing the Outlet Assembly, the Valve Gate and a three-actuator configuration is shown in FIG. 36. Note the three actuators are not in a single line. Each actuator extends and contracts axially and is nominally parallel to Surface 1 of the Outlet Assembly and Surface 2 of the Valve Gate. The three can extend an equal amount increasing or decreasing the gap, or differentially resulting in a rotation of Surface 2 of the Valve Gate about the X and or Y axis. Three or more sensors (not in a single line) measuring the distance between surface 1 and Surface 2. This data can be used to control the angle and distance between surface 1 and surface 2 to command the actuator length, resulting in the desired spacing. This can also compensate for manufacturing errors that result in surface 1 and surface 2 not being parallel.

When extended, a gap of length d(t) between these two surfaces results and gas flows at a rate Q_{Source}(t) from the reservoir at pressure P_{Reservoir} between surface 1 of the outlet assembly and surface 2 of the valve gate into the Outlet assembly and then out the Gas Source Outlet as shown by the Section A-A for the parallel and non-parallel valve gate configurations. Q_{Source}(t) is proportional to the gap, δ(t) between surface 1 and surface 2. One end of each actuator is attached to the outlet assembly with surface 1, and the other end is attached to surface 2 of the valve gate. The actuator extension, δ(t) is expected to range from 0 μm to 100 μm.

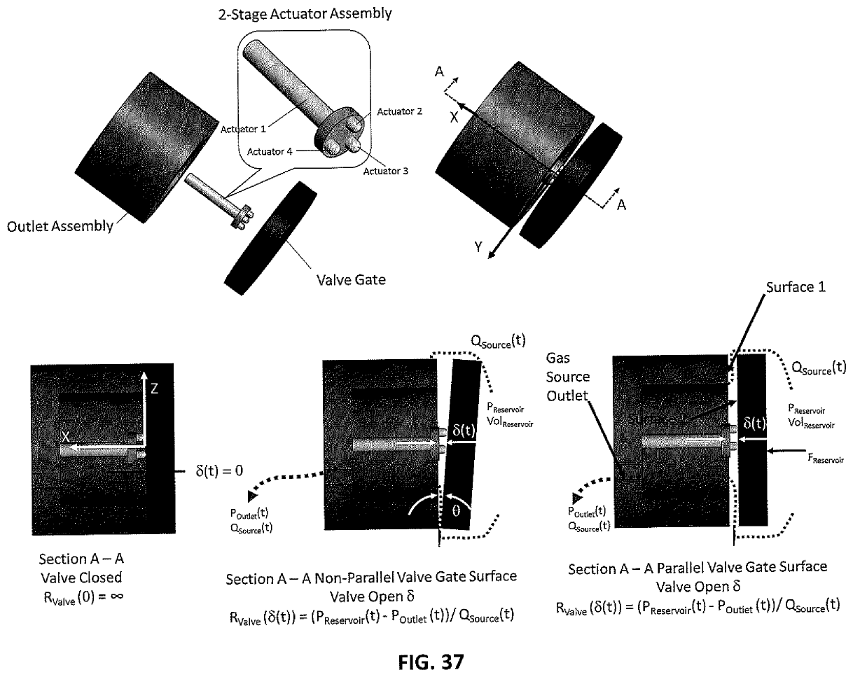

A simplified illustration showing the Outlet Assembly, the Valve Gate and a two-stage-actuator configuration is shown in FIG. 37. The two stage actuator has one long stroke actuator 1, that supports three actuators on top of it mounted on a plane. Actuator 1 and extend and contract axially along the X axis, and actuators 2-4 move together in tandem. Actuators 2-4 can move together resulting in a displacement of the Valve Gate surface 2 along the X axis, or differentially resulting in an angular motion of surface 2 about the X and or Y axis. Each actuator extends and contracts axially and is nominally parallel to Surface 1 of the Outlet Assembly and Surface 2 of the Valve Gate. Three or more sensors (not in a single line) measuring the distance between surface 1 and Surface 2. This data can be used to control the angle and distance between surface 1 and surface 2 to command the actuator length, resulting in the desired spacing. This can also compensate for manufacturing errors that result in surface 1 and surface 2 not being parallel.

Therefore, according to the principles described herein, A valve controlling gas flow rate by controlling the distance between two surfaces through which the gas passes may include a valve controlling gas flow rate by controlling the distance between two parallel surfaces through which the gas passes; and/or a valve controlling gas flow rate by controlling the distance between two flat and parallel surfaces through which the gas passes; and/or a valve controlling gas flow rate by controlling the average distance between two non-parallel surfaces through which the gas passes.

Disclosed is a method of controlling the distance between two surfaces with an actuator having the ability to lengthen or shorten in a single linear degree of freedom m. One end of the actuator is attached to structure associated with the first surface and the opposite end of the actuator to the second surface such that when the actuator lengthens or shortens, the corresponding distance between the surfaces increases or decreases. An actuator may be controlled open loop based on a pre-calibrated knowledge of distance between the two surfaces as a function of actuator length; and/or the actuator may be controlled closed loop based on feedback from: Gas flow rate between the surfaces; and/or pressure differential driving the gas flow rate; and/or measured distance between the two surfaces with one or more sensors; and/or reaction force through the actuator measured with a sensor.

Disclosed is a method of controlling the distance between two parallel flat surfaces with an actuator having the ability to lengthen or shorten in a single linear degree of freedom. One end of the actuator is attached to structure associated with to the first surface and the opposite end of the actuator to the second surface such that when it lengths or shortens, the corresponding distance between the surfaces increases or decreases. The actuator may be controlled open loop based on a pre-calibrated knowledge of distance between the two surfaces as a function of actuator length; and/or controlled closed loop based on feedback from gas flow rate between the surfaces; and/or pressure differential driving the gas flow rate; and/or measured distance between the two surfaces with one or more sensors; and/or reaction force through the actuator measured with a sensor.

Disclosed is a method of controlling the average distance between two non-parallel surfaces with an actuator having the ability to lengthen or shorten in a single linear degree of freedom. One end of the actuator is attached to structure associated with the first surface and the opposite end of the actuator to the second surface such that when it lengths or shortens, the corresponding distance between the surfaces increases or decreases. The actuator may be controlled open loop based on a pre-calibrated knowledge of distance between the two surfaces as a function of actuator length; and/or an actuator controlled closed loop based on feedback from: Gas flow rate between the surfaces; and/or pressure differential driving the gas flow rate; and/or measured distance between the two surfaces with one or more sensors; and/or reaction force through the actuator measured with a sensor.

Disclosed is a method of controlling the average distance between two non-parallel flat surfaces with an actuator having the ability to rotate about an axis parallel to both surfaces in a positive or negative direction, resulting in an increase or decrease in the average distance between the two surfaces. One side of the actuator is attached to structure associated with the first surface and the opposite side of the actuator to the second surface such that when the actuator angle increases or decreases the corresponding average distance between the surfaces increases or decreases. The actuator may be controlled open loop based on a pre-calibrated knowledge of distance between the two surfaces as a function of actuator angle; and/or controlled closed loop based on feedback from: gas flow rate between the surfaces; and/or pressure differential driving the gas flow rate; and/or measured distance between the two surfaces with a sensor; and/or reaction force through the actuator measured with one or more sensors; and/or reaction force through the actuator measured with a sensor

Disclosed is a method of controlling the distance between two surfaces with three or more actuators, none of them parallel to either surface) having the ability to lengthen or shorten in a single linear degree of freedom. One end of each actuator is attached to structure associated with the first surface and the opposite end of each actuator to the second surface such that when it lengths or shortens, the corresponding distance and or angle between the surfaces increases or decreases. Differentially controlling the length of each actuator allows for controlling the average distance and angle between the two surfaces. The actuators may be controlled open loop based on a pre-calibrated knowledge of distance and angle between the two surfaces as a function of actuator length; and/or may be controlled closed loop based on feedback from: gas flow rate between the surfaces; and/or pressure differential driving the gas flow rate; and/or measured distance and or angle between the two surfaces with sensors; and/or reaction force through the actuators measured with a sensor; and/or a method of controlling the distance between two surfaces with a 2-stage actuator where three or more actuators, none of them parallel to either surface) having the ability to lengthen or shorten in a single linear degree of freedom are placed on top of a fourth actuator. The forth actuator has the ability to lengthen or shorten, resulting is an axial displacement of the other three actuators. One end of the fourth actuator is attached to structure associated with the first surface and the opposite end is attached to a plane supporting the three or more actuators. The terminal end of each of the three actuators supported by the plane each attach to the second surface such that when it lengths or shortens, the corresponding distance and or angle between the two surfaces increase or decrease. Differentially controlling the length of each actuator on the plane allows for controlling the distance and angle between the two surfaces. The Actuators may be controlled open loop based on a pre-calibrated knowledge of distance and angle between the two surfaces as a function of actuator length; and/or may be controlled closed loop based on feedback from: gas flow rate between the surfaces; and/or pPressure differential driving the gas flow rate; and/or measured distance and or angle between the two surfaces with sensors; and/or reaction force through the actuators measured with a sensor.

Disclosed is a method of making the valve actuator to surface interface insensitive to changes in temperature through proper selection of material coefficient of thermal expansion properties of all components in the actuator load path between the first surface and the second surface includes selection of proper component length and coefficient of thermal expansion such that the change in distance between the actuator and surface interface is zero for an increase or decrease in temperature.

Disclosed is a method includes controlling gas flow into a pressurized nasal ventilation air chamber based on the flow rate into the air chamber from a gas source and the resulting differential pressure between the gas source and the air chamber, positioning a piston and controlling the vent position. Flow resistance of the vent is dependent on the piston position and associated vent in the air chamber. One side of the vent is coincident with the air chamber and the other side of the vent is coincident with the atmosphere. Flow from the air chamber through the vent to the atmosphere is dependent on the resulting flow resistance presented by the vent, and the air chamber pressure relative to the atmospheric pressure. The method may include a high flow rate from the gas source closes the vent by moving the piston and associated vent to close relative to the atmosphere due to the differential pressure between the higher gas source pressure and lower air chamber pressure across the piston surface. The flow resistance of the vent between the air chamber and atmosphere may be at a maximum in this position This results in no or minimal flow to the atmosphere, and preferential flow into the nares of the patient during the inhalation portion of a breathing cycle; and/or where a no or low flow rate from the gas source results in moving the piston in the opposite direction due to the differential pressure between the lower gas source pressure and higher air chamber pressure across the piston surface and opening the associated vent to the atmosphere. The flow resistance of the vent between the air chamber and atmosphere is at a minimum in this position. This results in flow to the atmosphere from the patient's nares during the exhalation portion of a breathing cycle; and/or where an intermediate flow rate from the gas source positions the piston to an intermediate position between being fully opened and closed due to the differential pressure between the higher gas source pressure and lower air chamber pressure across the piston surface. In this position the vent is partially opened relative to the atmosphere. The flow resistance of the vent between the air chamber and atmosphere is at a level adequate to maintain the Positive Expiratory End Pressure (PEEP) due to the resulting airflow from the gas source to the air chamber and from the pressure differential due to the flow through the vent from the air chamber to the atmosphere. This results in some flow to the atmosphere that is adequate to maintain the pressure level in the air chamber at or above nominally 5 CM H2O in in order to provide Positive Expiratory End Pressure (PEEP) to the patient's nares during the final expiratory portion of the breathing cycle where the expiratory flow from the patient alone is inadequate to maintain the PEEP.

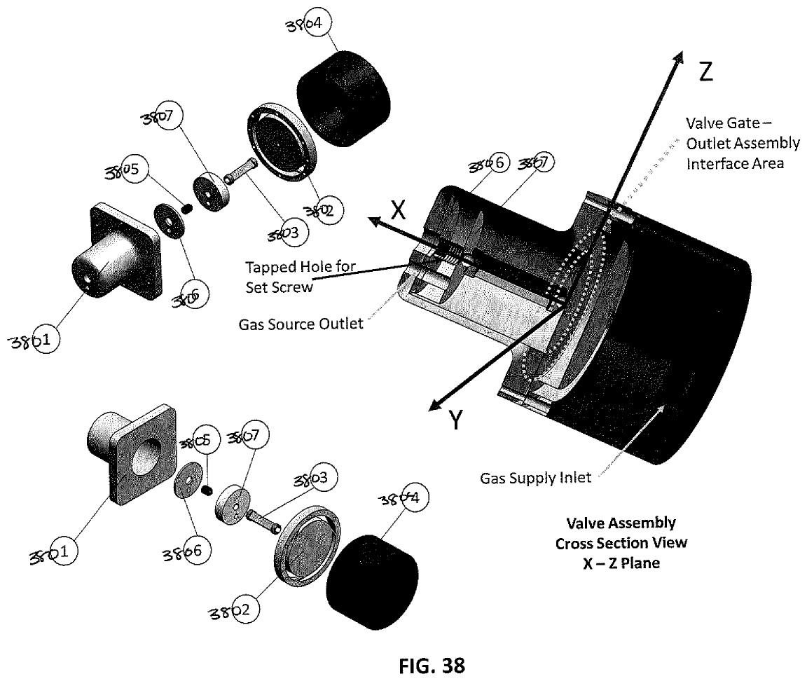

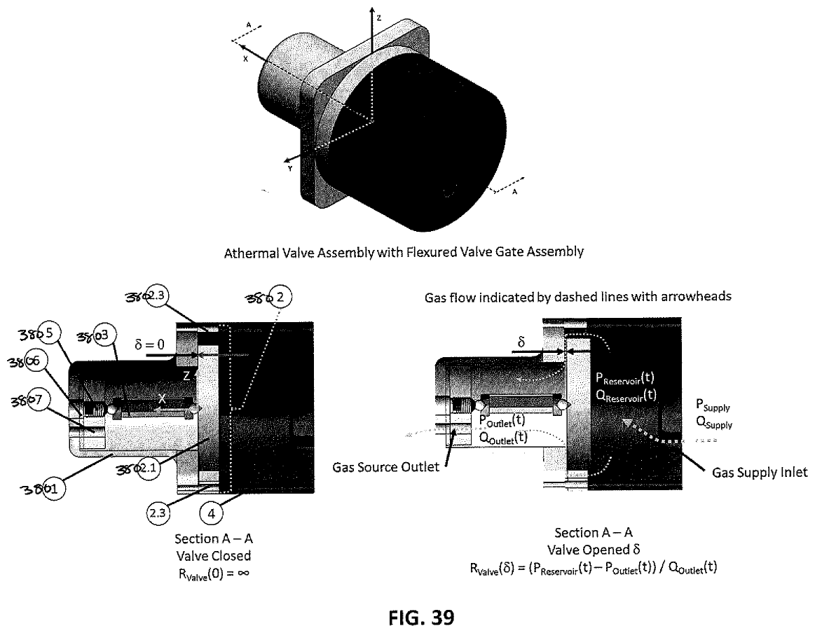

An Athermal Valve Assembly utilizing an integrated Valve Gate Assembly is shown in FIG. 38 and FIG. 39, with the actuator on the low-pressure side of the valve gate. The valve assembly includes an outlet assembly 3801, a valve gate assembly 3802, a valve gate 3802.1, one or more flexures 3802.2, a reaction ring 3802.3, an actuator assembly 3803, a reservoir housing 3804, a set screw 3805, an athermal spacer 3806 and an actuator reaction plate 3807.

The Outlet Assembly is the source low pressure side of the Valve Assembly. Flow is controlled by the size of the gap along the X axis between the Y-Z face on the +X axis side of Valve gate and the Y-Z face on the −X axis side of the Outlet assembly. Gas flows annularly in the Y-Z plane towards the X axis between these two surfaces. This interface area is highlighted in FIG. 38. The Gas Source outlet can interface with multiple gas interface circuits for patients including, but not limited to, a standard 15 mm or 22 mm circuit or another gas supply line. There is a hole in the end of the Outlet Assembly on the Y-Z face that allows for access to the set screw in the Actuator Reaction Plate.

The Valve Gate Assembly incudes three structurally integrated elements, the Valve Gate, flexure or flexures and reaction ring. The assembly is fabricated so that the Y-Z surface on the +X axis side of the assembly are coplanar and optically flat. When the actuator has no extension, δ=0.

The Y-Z face of the valve gate on the +X axis side is optically flat, resting against the optically flat Y-Z surface of the Outlet Assembly, −X side, when δ=0, presenting a nearly infinite flow resistance. The same Valve gate surface also interfaces with one end of the actuator assembly so when the actuator extends, it moves the valve gate along the X axis in the negative direction, allowing a gap between the valve gate and outlet assembly surfaces. The Valve Gate controls the source flow rate, Q_{Source}(t) based on its position along the X axis. The distance between the Valve Gate Face on the positive X axis of the Gate with surface area A_{Gate}, and the Outlet Assembly Face, δ, determines the flow resistance by creating a resistance channel between the outlet assembly surface and the valve gate Y-Z face. Reservoir pressure, P_{Reservoir}, provides a force in the +X direction, F_{P}, where F_{P}=P_{Reservoir} A_{Gate}.

The Valve Gate Assembly has one or more flexures that connect the Valve Gate and Reaction Ring. The flexure/flexures restrain valve gate motion in the Y-Z plane relative to the outlet assembly but allow for travel along the X axis.

The Y-Z face of the reaction ring on the +X axis side is optically flat, is mechanically interfaces with and fluidically seals against the optically flat Y-Z surface of the Outlet Assembly, −X side, providing the mechanical interface to the outlet assembly. Forces generated by valve gate motion due react through the flexure/flexures to the reaction ring. The Y-Z face of the reaction ring on the −X axis side mechanically interfaces with and fluidically seals against to the Y-Z surface of the Reservoir Housing, +X side.

The Actuator Assembly interfaces with the Valve Gate, positioning the Valve Gate along the X axis of the valve slide surface in the Y-Z plane, setting its position along the X axis. The assembly is a linear actuator that could be an electro-strictive material such as PZT or PMN, a magneto-strictive material, a screw drive or a voice coil drive or other linear drive mechanism. It's length and the resulting gate position is controlled under closed loop control based on the desired source flow rate, Q_{Source}(t) or flow source pressure, P_{Source}(t). I could also be driven under open loop control.

The Y-Z face, +X axis of the reservoir housing mechanically interfaces with and fluidically seals against the Y-Z surface, −X axis of the Valve Gate Assembly reaction ring. The Reservoir Housing contains the high-pressure side of the Valve Assembly. It acts as a gas reservoir, allowing the system to instantaneously supply a higher rate of gas flow than the average provided by the supply system, Q_{Supply}. The total volume of gas contained in the Reservoir Housing is governed by the Ideal gas law where n_{Reservoir}P_{Reservoir} V_{Reservoir}/RT_{Reservoir}. n is the number of moles of gas, R is the universal gas constant and T_{Reservoir} is the reservoir gas temperature. The Reservoir Housing has a Gas Supply Inlet can interface with a standard hospital O2 source or with any gas source or compressor.

The Set Screw resides in the Tapped Hole of the Outlet Assembly and interfaces with the actuator Assembly. Rotating the Screw drives the actuator assembly and valve gate in the X direction, setting the Initial position of the Valve Gate along the X axis.

The Athermal Spacer attaches to the Y-Z face on the −X axis side of the Outlet Assembly end and the Y-Z face on the +X axis side of the Actuator Reaction Plate. The CTE and thickness of the spacer are chosen to compensate for the CTE difference between the Actuator Assembly and the Outlet Assembly. There is no contact between the Athermal Spacer surfaces perpendicular to the Y-Z plane and the Outlet Assembly.

The Actuator Reaction Plate attaches to the Y-Z face on the −X axis side of Athermal Spacer and has the same CTE as the Outlet Assembly. There is also a tapped hole for the Set Screw and subsequent actuator position adjustment along the X axis. There is no contact between the Actuator Reaction Plate surfaces perpendicular to the Y-Z plane and the Outlet Assembly.

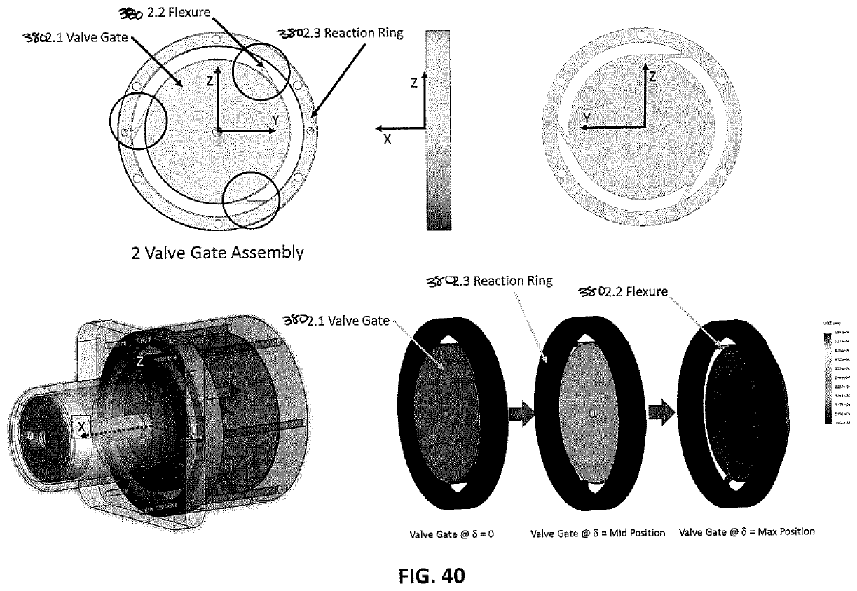

A major difference between this valve assembly and earlier versions presented is the merging of the valve gate and preload spring into a single valve gate assembly shown in FIG. 40. The advantages over this configuration and earlier configurations that the Reservoir had a valve gate shaft that constrained the valve gate in the Y-Z plane, for the shaft was contained in the shaft slot that was part of the valve gate. The shaft allowed for valve gate motion along the X axis. The spring assembly was concentric with the valve gate shaft reacting when compressed between the Y-Z surface of the valve gate on the −X axis side and the Reservoir Y-Z surface on the +X axis side. There is an issue in tolerance build up resulting in the valve gate shaft not being perpendicular to the outlet assembly surface in the Y-Z plane on the negative x axis side. It is possible that outlet assembly and valve gate surfaces are non-planer so as the valve goes to 0 and the surfaces contact, there is an opening due to the two surfaces being non-parallel preventing full closure.

The Valve gate assembly with an integral flexure set and reaction ring shown in FIG. 40 address the non-parallel surface issue mentioned earlier. The Valve Gate Assembly includes the Valve gate, one or more flexures that mechanically connect the valve gate to the reaction ring, constraining valve gate motion in the Y-Z plane but allowing for motion along the X axis, and the reaction ring. The reaction ring interfaces directly with the Outlet Assembly Y-Z surface on the −X axis side of the assembly, providing the mechanical ground. The lower portion of FIG. 40 shows Valve gate position at a zero displacement, mid displacement and maximum displacement relative to the reaction ring due to actuator extension.

The valve gate assembly is an integral component mechanically, where the Y-Z surface of the reaction ring and valve gate that interface with the mating outlet assembly surface (the valve gate outlet assembly interface region highlighted in FIG. 38, are lapped optically flat, nominally 0.06 um rms. The outlet assembly surface is also lapped optically flat. These surfaces are mated by machine screws that are inserted into the Y-Z surface facing the −X axis of the Reservoir housing and terminate in the outlet assembly, compressing the valve gate assembly against the outlet assembly Y- surface. When there is no extension of the actuator that separates the outlet assembly and valve gate surface, the valve is closed as shown in FIG. 29. When the valve gate is extended, there is a gap between the surfaces and flow from the reservoir housing to the outlet assembly results, also shown in FIG. 39. The flexure or flexures retain the valve gate in the Y-Z plane, allowing for travel along the X axis. There is no perpendicularity issue, for when the actuator displacement is zero, the valve gate returns to its original position and the two lapped adjoining surfaces (valve gate Y-Z plane on the +X axis side and the outlet assembly Y-Z surface on the −X axis side, are parallel and have a nominally zero gap to within optical tolerances and the valve is closed.

A cross-sectional view of the Valve Assembly with the actuator on the low-pressure side of the Valve Gate is shown in FIG. 39. Section A-A on the left of the figure shows the valve in a closed position, δ=0, and the valve flow resistance, R_{Valve}(0) infinite. The valve elements with callouts that were previously are also in the left-hand cross section. A cross sectional view on the right portion of FIG. 39 shows the valve assembly with the gate moved a distance δ, in the negative direction along the X axis. As a result, the valve resistance is no longer infinite and gas flows from the reservoir to the Gas Source outlet as shown by the dotted lines with arrows indicating the flow direction.

Gas flow Q_{Source}(t) travels from the Reservoir with pressure P_{Reservoir}(t) around the perimeter of the Valve Gate, along the gap of length L into the annulus of the Outlet Assembly, with pressure P_{Outlet}(t). An approximation for the flow relationship can be made by assuming the flow through a set of parallel plates. In this case, through the gap between the Valve Gate and Outlet Assembly.

The pressure drop from the reservoir housing to the outlet assembly is of the order of 50 PSI (0.34 MPa). Oxygen has a Joule-Thompson coefficient, μ, of approximately 5.18 K/MPa. This results in the average gas temperature dropping 1.8 K. Depending on the patient requirements, there may be a need to add or subtract energy to the gas flow, increasing or decreasing temperature, as it travels between the Outlet Assembly and Valve Gate Y-Z surfaces in order to increase the temperature.

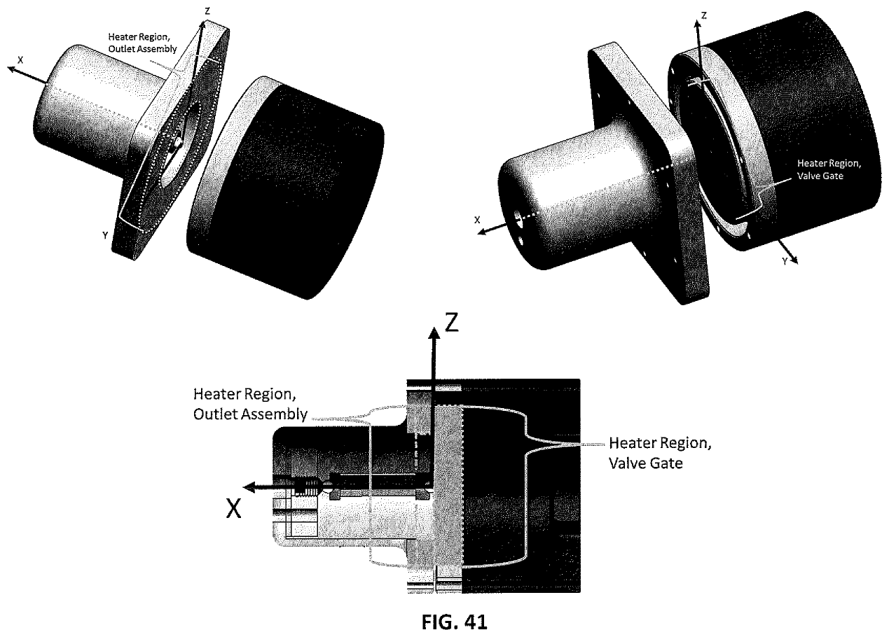

FIG. 41 shows the micro (μ) Gap (μGap) Valve with heater regions located at the Outlet Assembly and/or Valve Gate adding energy between the flow between these two Y-Z surfaces. The energy provided to these regions could be I²R resistive heating that conducts through the associated medium, then energy transfers convectively and/or radiatively to the air flow through the two surfaces. Thermo-electric heating/cooling or other methods of energy transfer to the air flow are also possible. The μGap valve thus allows for a method of adding or subtracting energy to the gas flowing between the Outlet Assembly and Valve Gate surfaces. The method includes adding or subtracting energy to the air flow through conductive then convective energy transfer from the Outlet Assembly Surface to the gas flow; and/or adding or subtracting energy to the air flow through conductive then convective energy transfer from the Valve Gate Surface to the gas flow; and/or adding or subtracting energy to the air flow through conductive then convective energy transfer from the Valve Gate Surface and Outlet Assembly Surface to the gas flow; and/or adding or subtracting energy to the air flow through conductive then radiative energy transfer from the Outlet Assembly Surface to the gas flow; and/or adding or subtracting energy to the air flow through conductive then radiative energy transfer from the Valve Gate Surface to the gas flow; and/or adding or subtracting energy to the air flow through conductive then radiative energy transfer from the Valve Gate Surface and Outlet Assembly Surface to the gas flow; and/or adding or subtracting energy to the air flow through conductive then convective energy transfer from the Valve Gate Surface to the gas flow and gas residing in the supply reservoir; and/or adding or subtracting energy to the air flow through conductive then radiative energy transfer from the Valve Gate Surface to the gas flow and gas residing in the supply reservoir.

In addition, provided is a valve controlling gas flow rate by controlling the distance between two surfaces through which the gas passes utilizing an integrated valve gate—flexure—reaction ring, valve gate assembly where the valve gate is the first surface and outlet assembly is the second surface. The assembly may include a valve gate assembly with integrated valve gate—flexure—reaction ring where the valve gate and reaction ring are co-planar and flat to optical tolerances in the neutral state where the actuator has zero extension. This optically co-planer valve gate assembly surface interfaces with a planer and optically flat outlet assembly surface. The flow resistance from the reservoir housing to the outlet assembly is nearly infinite with no flow occurring due to the pressure difference; and/or a condition where the actuator is extended, lifting the valve gate off the outlet assembly surface, allowing for flow from the reservoir housing to the outlet assembly due to the pressure difference between the two chambers by controlling the distance between two parallel surfaces through which the gas passes, with flow resistance proportional to the surface separation distance; and/or a condition where the actuator is initially extended, with the valve gate separated from the outlet assembly surface, allowing for flow from the reservoir housing to the outlet assembly due to the pressure difference between the two chambers by controlling the distance between two parallel surfaces. The actuator is retracted then retracted to the zero extension condition with the valve gate assembly with integrated valve gate—flexure—reaction ring where the valve gate and reaction ring are co-planer and flat to optical tolerances returns to the neutral state and once again is in contact with the planer and optically flat outlet assembly surface. The flow resistance from the reservoir housing to the outlet assembly is nearly infinite with no flow occurring due to the pressure difference.

A method of controlling the distance between two surfaces with an actuator is disclosed and has the ability to lengthen or shorten in a single linear degree of freedom. One end of the actuator is attached to structure associated with the first surface and the opposite end of the actuator to the second surface such that when the actuator lengthens or shortens, the corresponding distance between the surfaces increases or decreases, the method including an actuator controlled open loop based on a pre-calibrated knowledge of distance between the two surfaces as a function of actuator length; and/or an actuator controlled closed loop based on feedback from: Gas flow rate between the surfaces and/or Pressure differential driving the gas flow rate and/or measured distance between the two surfaces with one or more sensors; and/or reaction force through the actuator measured with a sensor.

The disclosure provides a method of making the valve actuator to valve gate surface interface insensitive to changes in temperature through proper selection of material coefficient of thermal expansion properties of all components in the actuator load path between the first surface and the second surface. The method includes selection of proper component length and coefficient of thermal expansion such that the change in distance between the actuator and surface interface is zero for an increase or decrease in temperature.

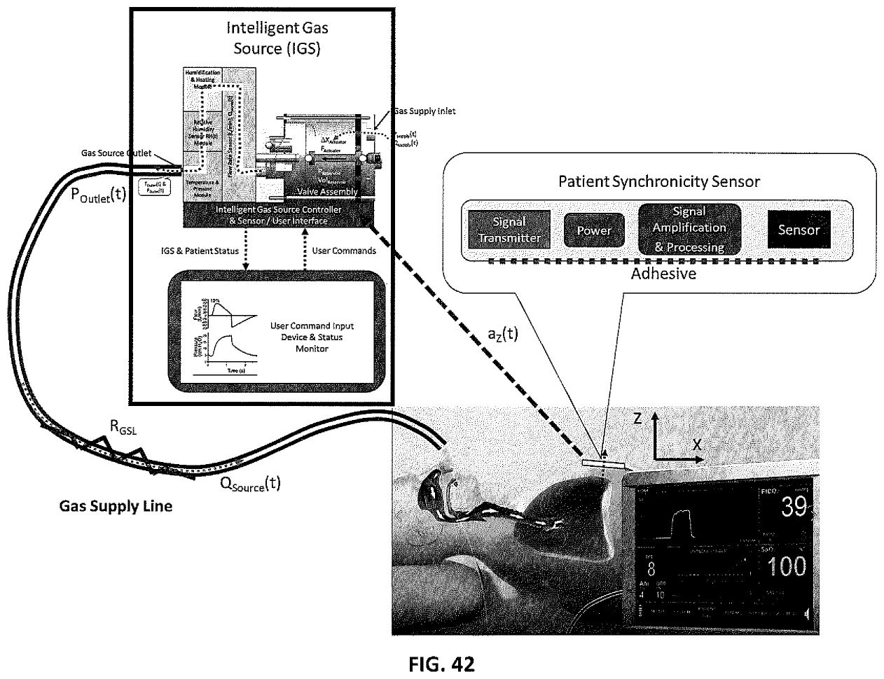

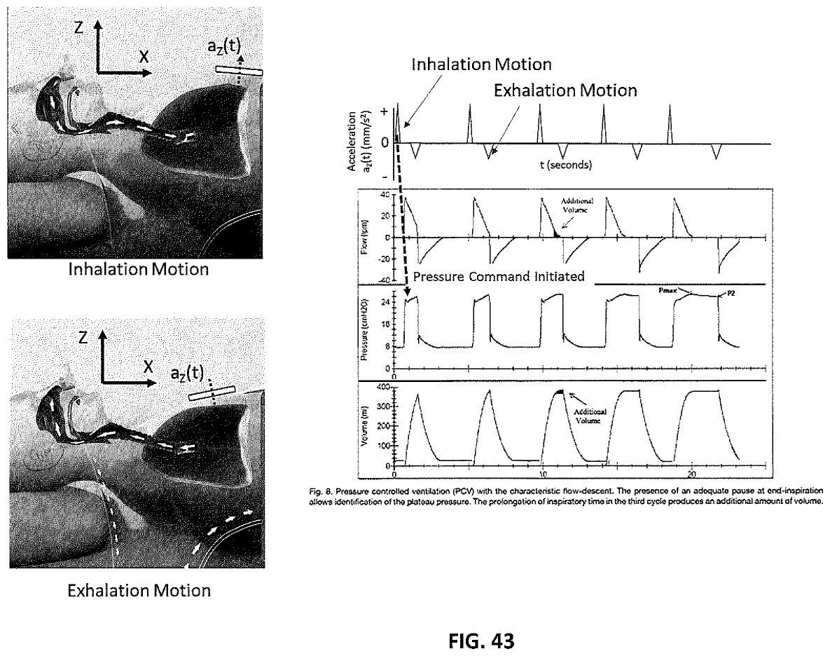

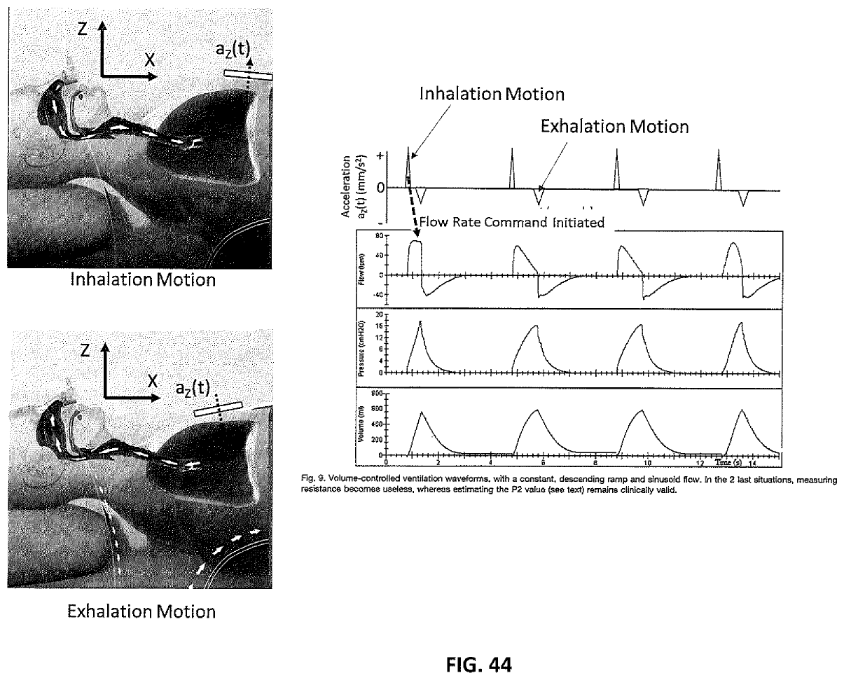

Patients interfacing with a ventilator often have an issue with the synchronization of ventilator breathing cycle. Current ventilators either set the breath per minute rate and hope the patient can keep up or attempt to synchronize utilizing pressure variations measured in the breathing patterns to time the supply of gas. What is proposed is a sensor that monitors patient chest, diaphragm and or acoustic signals that can be used to synchronize the flow of gas to the patient from the ventilators illustrated in FIG. 42. The Synchronicity Sensor is intended to provide information on what part of the inspiratory—expiratory cycle the patient is entering as illustrated in FIGS. 43 and 44. As the chest begins the inspiratory cycle, the chest and diaphragm will expand, resulting in an acceleration or sound due to air entering the lungs. During the expiratory, portion of the breathing cycle, this is reversed.

The Synchronicity Sensor is a package attached to the patient's chest or other area of the patient impacted by the breathing cycle. Elements of the sensor include a sensor is contained in the package that can measure one of the following parameters that changes due to the breathing cycle: Acceleration/velocity/position, Sound, Strain/stress, Power, a battery providing power to the system can be contained in the package, or an external power source attached via a wire can be provided. Sensor signal conditioning and amplification is required to prepare the signal for transmission to the intelligent gas source. A signal transmitter forwards the sensor signal either wirelessly or by wire to the intelligent gas source controller.

FIG. 43 is a plot as a function of time for the inspiratory/expiratory cycle of chest acceleration as measured by the Patient Synchronicity Sensor, Flow rate, Pressure and volume. Peak acceleration is initially measured as the patient begins inspiration and the chest rises. This sensor signal is used as a trigger to drive the commanded pressure supplied by the intelligent gas source. This cycle is repeated indefinitely.

FIG. 44 is a plot as a function of time for the inspiratory/expiratory cycle of chest acceleration as measured by the Patient Synchronicity Sensor, Flow rate, Pressure and volume. Peak acceleration is initially measured as the patient begins inspiration and the chest rises. This sensor signal is used as a trigger to drive the commanded flow rate supplied by the intelligent gas source. This cycle is repeated indefinitely.

The disclosed synchronicity sensor thus can be designed to work with any of the devices disclosed herein, including the pressurized nasal ventilator apparatus and intelligent gas source. A sensor that monitors physical properties of the inhalation and exhalation cycle of a patient for the purpose of triggering a ventilator to provide commanded pressure or gas flow to a patient from a ventilator, the physical properties including: Acceleration, velocity or position of the chest or diaphragm; and/or Body surface strain due to the inhalation/exhalation cycle; and/or Acoustical noise associated with the inhalation/exhalation cycle; and/or The sensor attached to the patient's body surface. The above described sensor combined with a gas source, including, but not limited to any permutation or combination of elements forming the intelligent gas source described herein. The sensor may be a group of individual sensors for monitoring the physical properties. A method of using any of the above described sensors and/or gas sources.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

While various embodiments of the present invention have been described above, it should be understood that they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments, but should be defined only in accordance with the following claims and their equivalents.

1. A nasal ventilator assembly, comprising:
a nasal interface comprising at least one opening for fluid communication with the nares of a patient;
an air chamber in fluid communication with the nasal interface, a gas supply port and an end tidal sample port, a vent assembly comprising:
a vent housing comprising at least one housing vent hole in fluid communication with ambient atmosphere; and
a piston assembly comprising a piston, a spring, a piston end wall operatively connected with the piston and the spring and a piston driven wall operatively connected with the piston and the spring, the piston driven wall comprising at least one piston wall vent hole in fluid communication with the air chamber,
wherein motion of the piston is caused by inhalation and exhalation of a patient to move the housing vent hole to align with the piston wall vent hole upon exhalation of the patient to cause the air chamber to be in fluid communication with the ambient atmosphere upon exhalation of the patient and to be fluidically sealed from the ambient atmosphere during inhalation of the patient.

2. The nasal ventilator assembly of the claim 1, wherein the piston assembly is inserted into the vent housing, with the piston driven wall and the piston end wall moveable within the vent housing according to movement of the piston and the spring.

3. The nasal ventilator assembly of claim 1, wherein the vent housing has a front vent wall, the front vent wall including the at least one housing

4. The nasal ventilator assembly of claim 1, wherein the piston assembly comprises a slide rail and the vent housing comprises a slide rail opening corresponding to the slide rail, such that the slide rail is moveable within the slide rail opening, thus constraining movement of the piston assembly within the vent housing.

5. The nasal ventilator assembly of claim 4, wherein the piston driven wall comprises the slide rail and the vent housing comprises the slide rail opening.

6. The nasal ventilator assembly of claim 1, wherein the vent housing includes a plurality of housing vent holes and the piston driven wall comprises a corresponding plurality piston wall vent holes.

7. The nasal ventilator assembly of claim 1, wherein the housing vent hole has a star cross section and the piston wall vent hole has a star cross section.

8. The nasal ventilator assembly of claim 1, wherein the housing vent hole has a substantially circular cross section and the piston wall vent hole has a substantially circular cross section.

9. The nasal ventilator assembly of claim 1, wherein the housing vent hole has a substantially rectangular cross section and the piston wall vent hole has a substantially rectangular cross section.

10. The nasal ventilator assembly of claim 1, wherein the piston driven wall includes an additional vent hole.

11. The nasal ventilator assembly of claim 10, wherein the additional vent hole has a substantially circular configuration having a cross sectional area smaller than the piston wall vent hole.

12. The nasal ventilator assembly of claim 1, wherein the piston end wall comprises a piston opening therethrough.

13. The nasal ventilator assembly of claim 10, wherein a location of the piston opening corresponds to the gas supply port in fluid communication with the air chamber.

14. A gas source controller for pressurized oxygenation of a patient, comprising:
a valve assembly, comprising:
a valve gate assembly comprising a valve gate;
an actuator assembly operatively connected to the valve gate to cause motion of the valve gate in a predetermined direction;
a reservoir housing comprising a gas reservoir for containing a predetermined volume of gas therein,
an end cap having a gas inlet therethrough, and
an actuator control operatively coupled to the actuator assembly;
an outlet assembly comprising a gas sensor and a gas source outlet; and
a user interface for receiving control signals for driving the actuator assembly.

15. The gas source controller of claim 14, wherein the valve assembly further comprises a gasket between the outlet assembly and the reservoir housing.

16. The gas source controller of claim 14, further comprising an actuator retainer gasket between the reservoir housing and the end cap.

17. The gas source controller of claim 14, wherein the actuator assembly comprises a linear actuator controllable according to desired flow rate, or flow source pressure.

18. The gas source controller of claim 14, further comprising a gap between a face of the valve gate and a face of the outlet assembly such that gas flows annularly between face of the valve gate and the face of the outlet assembly.

19. The gas source controller of claim 14, wherein the gas sensor is selected from at least one of a flow rate sensor, a relative humidity sensor, a temperature sensor and a pressure sensor.

20. The gas source controller of claim 14, the outlet assembly further comprising at least one of a humidification module, a heating module, a temperature module and a pressure module for changing properties of gas flowing from the valve assembly to the gas source outlet.

21.-33. (canceled)
